(19) United States
(12) Patent Application Publication
(10) Pub. No.: US 2023/0302084 A1
(43) Pub. Date: Sep. 28, 2023

(54) METHODS AND COMPOSITIONS FOR TREATING PERIODONTAL DISEASE

(71) Applicant: University of Louisville Research Foundation, Inc., Louisville, KY (US)

(72) Inventors: Donald R. Demuth, Louisville, KY (US); Jill M. Steinbach-Rankins, Louisville, KY (US)

(21) Appl. No.: 18/020,235

(22) PCT Filed: Aug. 6, 2021
(86) PCT No.: PCT/US2021/045122

§ 371 Date: Feb. 7, 2023

Related U.S. Application Data
(60) Provisional application No. 63/062,933, filed on Aug. 7, 2020.

(51) Int. Cl. A61K 38/16; A61P 31/04; A61K 9/16; A61K 9/00

(52) U.S. Cl. CPC A61K 38/164; A61P 31/04; A61K 9/1647; A61K 9/0056; D01D 5/003

(57) ABSTRACT

Compositions and methods of treating periodontitis are provided. Such compositions and methods typically include a BAR polypeptide and a delivery vehicle.

### CROSS-REFERENCE TO RELATED APPLICATIONS

This application claims the benefit of priority under 35 U.S.C. § 119 to U.S. Provisional Application Ser. No. 63/062,933, filed Aug. 7, 2020, the entirety of which is incorporated herein by reference.

### FEDERALLY SPONSORED RESEARCH OR DEVELOPMENT

This invention was made with government support under DE023206 and DE025345 awarded by National Institutes of Health—National Institute for Dental and Craniofacial Research and GM125504 awarded by National Institutes of Health—National Institute of General Medical Sciences. The government has certain rights in the invention.

### TECHNICAL FIELD

This disclosure generally relates to methods and compositions for treating periodontal disease.

### BACKGROUND

Active agents targeting key bacterial interactions that initiate biofilm formation in the oral cavity may alter periodontitis progression. To date, however, specifically-targeted prophylactic and treatment strategies have been limited. Previously, a peptide, BAR (SspB Adherence Region), was developed that inhibits oral P. gingivalis/S. gordonii biofilm formation in vitro and in vivo, and BAR nanoparticles that increase BAR effectiveness via multivalency and prolonged delivery. Limited BAR loading and nanoparticle retention in the oral cavity can result in inadequate release and efficaciousness. Given this, an effective delivery platform that can release concentrations of BAR suitable for, e.g., once-daily or twice-daily applications may offer an alternative that enhances loading, ease of administration, and retention in the oral cavity.

### SUMMARY

Compositions and methods of treating periodontitis are described herein. As described herein, such compositions and methods typically include a BAR polypeptide and a delivery vehicle.

In one aspect, compositions comprising a BAR polypeptide encapsulated within a nanoparticle are provided.

In some embodiments, the nanoparticle is comprised of at least one hydrophobic polymer and/or at least one hydrophilic polymer. In some embodiments, the at least one hydrophobic polymer is selected from poly(lactic-co-glycolic) acid (PLGA), poly(L-lactic acid) (PLLA), polycaprolactone (PCL), polyurethane, silicone, and combinations thereof. In some embodiments, the at least one hydrophilic polymer is selected from polyethylene oxide (PEO), polyvinyl pyrrolidone, polyvinyl alcohol, and combinations thereof.

In some embodiments, the nanoparticles have an average diameter of about 50 nm to about 100 nm.

In some embodiments, the surface of the nanoparticle is modified. Representative modifications include, without limitation, CafA polypeptides and antibodies or Fab fragments that are reactive toward Streptococcal surface proteins or Streptococcal carbohydrates. In some embodiments, the modification is selected from an antibiotic, an antiseptic, an antimicrobial peptide, and combinations thereof. In some embodiments, the modification is CafA polypeptides bound to palmitate. In some embodiments, the density of the modification on the surface of the nanoparticle is about 3 μg modifier/mg polymer to about 40 μg modifier/mg polymer (e.g., about 20 μg modifier/mg polymer).

In some embodiments, the composition is formulated for delivery to the oral cavity. In some embodiments, the composition is formulated for delivery as dental strips, as gum, or as a gel. In some embodiments, the composition is formulated for rapid release.

In some embodiments, the BAR polypeptide has the sequence shown in SEQ ID NO:1.

In another aspect, methods of preventing or reducing an interaction between Porphyromonas gingivalis and streptococci spp. in the oral cavity of an individual are provided. Such methods typically include administering the composition of any of the claims above to the oral cavity of the individual, thereby preventing or reducing the interaction between Porphyromonas gingivalis and streptococci spp. in the oral cavity of the individual.

In some embodiments, the composition releases inhibitory concentrations of BAR polypeptide for at least 2 hours (e.g., at least 4 hours, at least 6 hours, at least 10 hours, at least 12 hours, or at least 18 hours).

In some embodiments, the composition is administered once per day. In some embodiments, the composition is administered twice per day.

In still another aspect, methods of making the composition described herein are provided. Such methods typically include combining a solution comprising hydrophilic polymers and/or hydrophobic polymers with the BAR polypeptides under conditions in which the solution forms nanoparticles, wherein the BAR polypeptides are encapsulated by the nanoparticles.

In some embodiments, such methods further include the step of modifying the nanoparticle. A representative modification is a CafA polypeptide.

In yet another aspect, compositions are provided that include electrospun fibers comprised of at least one hydrophobic polymer and/or at least one hydrophilic polymer; and a SspB Adherence Region (BAR) polypeptide from a streptococcal antigen I/II protein.

In some embodiments, the at least one hydrophobic polymer is selected from poly(lactic-co-glycolic acid) (PLGA), poly(L-lactic acid) (PLLA), polycaprolactone (PCL), polyurethane, silicone, and combinations thereof. In some embodiments, the at least one hydrophilic polymer is selected from polyethylene oxide (PEO), polyvinyl pyrrolidone, polyvinyl alcohol, and combinations thereof. In some embodiments, the hydrophilic polymer is PLGA.

In some embodiments, the ratio of hydrophobic to hydrophilic polymer is at least 49:51. In some embodiments, the ratio of hydrophobic to hydrophilic polymer is about 20:80. In some embodiments, the ratio of hydrophobic to hydrophilic polymer is about 10:90. In some embodiments, the at least one hydrophobic polymer or the at least one hydrophilic polymer is present in an amount of about 10%-18% w/w (e.g., 12%-15% w/w).

In some embodiments, the diameter of the fibers is about 0.5 μm to about 1.5 μm (e.g., about 0.7 μm to about 1.3 μm).

In some embodiments, the BAR polypeptide comprises residues 1167-1193 of the SspB protein sequence of S. gordonii. In some embodiments, the BAR polypeptide has the sequence shown in SEQ ID NO:1. In some embodiments, the BAR polypeptide is present at an amount of 1% w/w relative to the at least one hydrophobic polymer and/or the at least one hydrophilic polymer.

In some embodiments, the composition is configured for delivery to the oral cavity. In some embodiments, the composition is configured for delivery as dental strips or gum or a gel. In some embodiments, the composition is configured for rapid release.

In another aspect, methods of preventing or reducing an interaction between Porphyromonas gingivalis and streptococci spp. in the oral cavity of an individual are provided. Such methods typically include administering a composition as described herein to the oral cavity of the individual, thereby preventing or reducing the interaction between Porphyromonas gingivalis and streptococci spp. in the oral cavity of the individual.

In some embodiments, the composition is administered once per day. In some embodiments, the composition is administered twice per day.

In still another aspect, methods of making EF compositions as described herein are provided. Such methods typically include combining a solution comprising at least one hydrophobic polymer and/or at least one hydrophilic polymer with a BAR polypeptide with to produce a polymer-BAR polypeptide solution; and electrospinning the polymer-BAR solution to produce BAR polypeptide-incorporated fibers.

In some embodiments, the amount of BAR polypeptide in the solution is about 1.0 μM to about 5.0 μM. In some embodiments, the amount of BAR polypeptide in the solution is about 1.3 μM to about 3.4 μM. In some embodiments, the amount of BAR polypeptide in the solution is about 1% by weight.

In one aspect, compositions for treating periodontitis are provided. Such compositions generally include electrospun fibers comprised of at least one hydrophobic polymer and/or at least one hydrophilic polymer, where the at least one hydrophobic polymer is selected from poly(lactic-co-glycolic) acid (PLGA), poly(L-lactic acid) (PLLA), polycaprolactone (PCL), polyurethane, silicone, and combinations thereof, the at least one hydrophilic polymer is selected from polyethylene oxide (PEO), polyvinyl pyrrolidone, polyvinyl alcohol, and combinations thereof, and a polypeptide comprising a SspB Adherence Region (BAR) from a streptococcal antigen I/II protein (referred to herein as a BAR polypeptide).

In some embodiments, the composition is configured for delivery to the oral cavity (e.g., as dental strips. as gum). In some embodiments, the composition is configured for rapid release.

In some embodiments, the ratio of hydrophobic to hydrophilic polymer is at least 49:51. In some embodiments, the ratio of hydrophobic to hydrophilic polymer is about 20:80. In some embodiments, the ratio of hydrophobic to hydrophilic polymer is about 10:90.

In some embodiments, the hydrophilic polymer is PLGA.

In another aspect, methods of treating periodontitis in an individual are provided. Such methods generally include administering a composition as described herein to the individual, thereby treating periodontitis in the individual.

In some embodiments, the composition is formulated for delivery to the oral cavity (e.g., as dental strips, as gum). In some embodiments, the composition is administered twice per day. In some embodiments, the composition is formulated for rapid release.

In some embodiments, the ratio of hydrophobic to hydrophilic polymer is at least 49:51. In some embodiments, the ratio of hydrophobic to hydrophilic polymer is about 20:80. In some embodiments, the ratio of hydrophobic to hydrophilic polymer is about 10:90.

In some embodiments, the hydrophobic polymer is PLGA.

In still another aspect, compositions that include nanoparticles encapsulating a BAR polypeptide are provided.

In some embodiments, the nanoparticle is comprised of at least one hydrophobic polymer and/or at least one hydrophilic polymer. Representative hydrophobic polymers include, without limitation, poly(lactic-co-glycolic) acid (PLGA), poly(L-lactic acid) (PLLA), polycaprolactone (PCL), polyurethane, silicone, and combinations thereof. Representative hydrophilic polymers include, without limitation, polyethylene oxide (PEO), polyvinyl pyrrolidone, polyvinyl alcohol, and combinations thereof.

In some embodiments, the nanoparticle is modified (e.g., an antibiotic, an antiseptic, an antimicrobial peptide, and combinations thereof such as, without limitation, CafA and antibodies or Fab fragments that are reactive toward Streptococcal surface proteins or carbohydrates).

In some embodiments, the composition is formulated as a gel.

In still another aspect, methods of treating periodontitis in an individual are provided. Such methods typically include administering a composition as described herein to the individual, thereby treating periodontitis in the individual.

In some embodiments, the composition is administered once daily or twice daily.

In some embodiments, the composition releases inhibitory concentrations of BAR polypeptide for at least 8 hours (e.g., at least 12 hours, at least 18 hours).

In some of the embodiments described herein, the BAR polypeptide has the sequence shown in SEQ ID NO:1 or has a sequence having at least 90% sequence identity (e.g., at least 91%, 92%, 93%, 94%, 95%, 96%, 97%, 98%, 99% or 100%) to SEQ ID NO:1.

Unless otherwise defined, all technical and scientific terms used herein have the same meaning as commonly understood by one of ordinary skill in the art to which the methods and compositions of matter belong. Although methods and materials similar or equivalent to those described herein can be used in the practice or testing of the methods and compositions of matter, suitable methods and materials are described below. In addition, the materials, methods, and examples are illustrative only and not intended to be limiting. All publications, patent applications, patents, and other references mentioned herein are incorporated by reference in their entirety.

### DESCRIPTION OF DRAWINGS

Part A-Rapid Release Polymeric Fibers for Inhibition of Porphyromonas gingivalis and Streptococcus gordonii Biofilms

FIG. 1A are SEM images of 1% w/w BAR PLGA, PLLA, and PCL non-blended fibers.

FIG. 1B are SEM images of 40:60, 20:80, and 10:90 1% w/w BAR blended PLGA:PEO, PLLA:PEO and PCL:PEO fibers.

FIG. 2A-2D are graphs showing the average diameters of electrospun fibers measured from SEM images, using ImageJ. Non-blended (2A) and 40:60 blended (2B), 20:80 blended (2C), and 10:90 blended (2D) PLGA:PEO, PLLA:PEO and PCL:PEO with 1% w/w BAR fibers. Error bars represent the mean±the standard deviation (n=3) of three independent experiments.

FIG. 3A-3B are graphs showing the cumulative release of F-BAR from 1% w/w F-BAR non-blended (100:0) PLGA, PLLA and PCL fibers. The cumulative release is reported as μg F-BAR per mg of fiber (3A), and percent of total loaded F-BAR (3B). PLGA showed the greatest release of incorporated BAR among the non-blended formulations at 24 hr. Error bars represent the mean±the standard deviation (n=3) of three independent experiments.

FIG. 4A-4C are graphs showing the cumulative release of F-BAR from 1% w/w F-BAR blended PLGA:PEO, PLLA:PEO and PCL:PEO fibers 40:60 (4A), 20:80 (4B), and 10:90 (4C). The cumulative release is reported as the total quantity of F-BAR released on the left (μg F-BAR per mg of fiber), and as the percent of total loaded F-BAR on the right. Error bars represent the mean±the standard deviation (n=3) of three independent experiments.

FIG. 5A-5C are graphs showing the cumulative release of F-BAR from the non-blended and PEO-blended formulations as a function of hydrophobic polymer type PLGA (5A), PLLA (5B), or PCL (5C) and PEO ratio in each blend. The release of encapsulated BAR increases with an increase in PEO fraction. PLGA and PEO blends exhibit the most significant and rapid F-BAR release, relative to PLLA and PCL blends. For all polymer types, the 10:90 blends show the greatest release of BAR as compared to the 20:80 and 40:60 formulations at any given time point. PLGA:PEO (10:90) fibers provide the highest amount of BAR release across formulations. Data represent the mean±standard deviation (n=3) of three independent experiments.

FIG. 6 are photographs showing that BAR-incorporated PLGA:PEO (10:90) EFs prevent P. gingivalis adherence to S. gordonii. Biofilms were visualized with confocal microscopy and the ratio of green (P. gingivalis) to red (S. gordonii) fluorescence in z-stack images was determined using Volocity image analysis software. Each grid represents 21 μm.

FIG. 7 are photographs showing that BAR-incorporated PLGA:PEO (10:90) EFs disrupt pre-established P. gingivalis-S. gordonii biofilms. Biofilms were visualized with confocal microscopy and the ratio of green (P. gingivalis) to red (S. gordonii) fluorescence in z-stack images was determined using Volocity image analysis software. Each grid represents 21 μm.

FIG. 8A-8B are graphs showing biofilm inhibition (prevention) (A) and disruption (treatment) (B), as a function of different concentrations of BAR-incorporated PLGA:PEO (10:90) EFs and free BAR (3 μM). Data represent the mean standard deviation (n=6) of six independent experiments.

FIG. 9A is a graph showing the hemolytic activity of free BAR or 10:90 PLGA:PEO BAR-EFs (1.3, 3.4 μM) assessed after administration to sheep erythrocytes for 3 hr. Free BAR and BAR-EFs showed negligible hemolysis for sheep erythrocyte relative to release from H₂O-treated cells (****, P≤0.0001).

FIG. 9B is a graph showing the effect of free BAR and BAR-EFs (1.3, 3.4 μM) on TIGK cell viability. Free BAR and BAR-EFs were non-toxic, relative to cells treated with DMSO (****, P≤0.0001).

FIG. 9C is a graph showing the metabolic activity of cells treated with free BAR or BAR-EFs (1.3, 3.4 μM). BAR-EF (3.4 μM) treated cells showed decreases in ATP levels relative to medium-only treated cells, while TIGK cells treated with staurosporine demonstrated lower ATP levels than the cells treated with medium-only, free BAR, and BAR-EFs (****, P≤0.0001).

FIG. 9D is a graph showing that TIGK cell membrane integrity was assessed after administration of free BAR or BAR-EFs (1.3, 3.4 μM) by measuring LDH release levels. None of free BAR or BAR-EF (1.3, 3.4 μM) treated cells released a significant level of LDH relative to medium-only treated cells. Staurosporine-treated cells demonstrated significantly elevated LDH levels (****, P≤0.0001). Data represent the mean±standard deviation (n=5) of five independent experiments.

FIG. 10 is a graph showing the number of AP sites per 100,000 base pairs of genomic DNA obtained from TIGK cells treated with free BAR or BAR-EFs (1.3, 3.4 μM). Negligible changes in the number of AP sites were observed in cells treated with free BAR or 10:90 PLGA:PEO BAR-EFs (1.3, 3.4 μM), relative to medium-only treated cells. However, TIGK cells treated with 2 mM H₂O₂ demonstrated significantly (***, P≤0.001) higher numbers of AP sites relative to the untreated control, free BAR and BAR-EF (1.3, 3.4 μM) treated cells. Data represent the mean standard deviation (n=3) of three independent experiments.

### Part B—Assessment of Targeted BAR-Encapsulated NPs Against Oral Biofilms

FIG. 11A is a schematic representation of P. gingivalis-S. gordonii adhesion mediated via the interaction of major (fimA) and minor fimbriae (Mfal) of P. gingivalis with glyceraldehyde 3-phosphate dehydrogenase (GAPDH) and motifs on SspA/B proteins of S. gordonii, respectively.

FIG. 11B is a schematic representation of type 2 fimbriae of A. oris.

FIG. 11C is a schematic showing that surface modification of nanoparticles with CafA protein aids in directing nanoparticles to S. gordonii for targeted delivery of BAR peptide.

FIG. 12A-12B are SEM images of CafA-modified (12A) and unmodified (12B) BAR-encapsulated PLGA NPs. Images are representative of a minimum of 3 independent samples, with n>500 NPs assessed in total. Scale bars represent 1 μm.

FIG. 13 is a graphs showing CafA surface density as a function of CafA input concentration. The total amount of CafA conjugated to the NP surface was determined using the microBCA assay. The amount of CafA conjugated to the NP surface varied directly with the input concentration of CafA used during synthesis. Data represent the mean NP-associated CafA±standard deviation.

FIG. 14A-14B are graphs showing the cumulative release of BAR as a function of mass (μg BAR per mg NP) (14A) and percent of total BAR encapsulated (14B). Error bars represent the mean BAR released±standard deviation. Note that, in some cases, the error bars are smaller than the data point markers.

FIG. 15A is a graph showing that, after one hour of initial binding (t=0), a 14.4-fold higher concentration of CafA-modified NPs bound to S. g. relative to unmodified NPs, and this difference in concentration was maintained for up to 12 hr.

FIG. 15B is a graph showing that, after 12 hr, 45% of the administered CafA-modified NPs remain bound to S. g.

FIG. 15C is a graph showing that CafA binds to GalNAcβ1-3Gal motif of the receptor polysaccharides found only on commensal oral streptococci such as S. gordonii and S. oralis, and demonstrates minimal binding to bacteria lacking this motif Here, CafA-modified NPs bound to S. gordonii DL-1 and S. oralis SO34 at a higher concentration relative to S. mutans KPSP2, P. gingivalis ATCC 33277, and A. actinomycetemcomitans 652. Asterisks denote statistical significance between two groups (* p<0.01, ** p<0.001, *** p<0.0001, **** p<0.00001).

FIG. 16A-16E show the adherence inhibition of P. gingivalis to S. gordonii after different durations of administration. CafA-modified BAR NPs inhibited P. gingivalis adherence for over 12 hr relative to CafA-modified blank NPs (control). Biofilms were visualized using confocal microscopy and the ratio to green (P. g.) to red (S. g.) fluorescence in z-stack images was determined using Volocity software.

FIG. 16F is a graph showing CafA-modified BAR NP mediated inhibition of P. gingivalis adherence to S. gordonii at different time points in a dual-species biofilm. Error bars represent the mean inhibition±standard deviation.

FIG. 17A is a graph of TIGK cell viability assessed after CafA-modified BAR NPs or unmodified BAR NPs administration. CafA-modified BAR NPs and unmodified BAR NPs were non-toxic, relative to cells treated with DMSO (***, p≤0.001).

FIG. 17B is a graph showing that ATP levels from CafA-modified BAR NPs and unmodified BAR NPs (1.3, 3.4 μM) treated cells showed nearly the same level of ATP concentration of control cells (treated with medium only), while ATP levels in the staurosporine-treated cells were significantly lower than the control (treated with medium only), CafA-modified BAR NPs, and unmodified BAR NPs-treated cells (*** p≤0.001).

FIG. 17C is a graph showing that no significant release of LDH was observed from TIGK cells treated with CafA-modified BAR NPs and unmodified BAR NPs, relative to control cells. Staurosporine-treated cells demonstrated significantly elevated LDH levels (***, p≤0.001). Data represent the mean±standard deviation (n=3).

### DETAILED DESCRIPTION

Periodontal disease is a group of chronic inflammatory diseases that are globally prevalent, affecting over 65 million adults in the U.S., with increased incidence in developing countries. Moreover, the prevalence and severity of periodontal disease has been shown to increase from 47 to 64% in adults from age 30 to 65. Advanced periodontal disease (subgingival pocket depths>6 mm) occurs in up to 110% of adults worldwide, and is a chronic, irreversible inflammatory disease that results in destruction of connective tissue, vascular proliferation, and alveolar bone resorption. Porphyromonas gingivalis is strongly associated with chronic adult periodontitis and has been considered to be a key pathogen that may promote disease by perturbing host-microbe homeostasis, leading to uncontrolled inflammation. In addition to its localized oral effects, periodontal disease has been associated with systemic diseases, such as cardiovascular disease, type 2 diabetes mellitus, low birth weights, and osteoporosis.

While the primary niche of P. gingivalis is the anaerobic environment of the subgingival pocket, P. gingivalis initially colonizes the oral cavity by interacting with Gram-positive commensal streptococci in the supragingival environment. The initial adhesion of P. gingivalis to streptococci (e.g., S. gordonii) in the supragingival environment is mediated via the interaction of the major (fimA) and minor fimbriae (Mfal) of P. gingivalis with glyceraldehyde 3-phosphate dehydrogenase (GAPDH) and motifs on streptococcal antigen I/II protein (e.g., SspA/SspB) proteins of S. gordonii, respectively (FIG. 11A). Blocking these initial P. gingivalis/S. gordonii interactions may limit the supragingival colonization of P. gingivalis and subsequent subgingival biofilm formation, providing early stage targets for therapeutic intervention.

Delivery vehicles that localize the delivery and maintain the stability of specifically-targeted biologics, such as BAR peptide, may offer improved functional activity, thereby enhancing the therapeutic efficacy. Delivery platforms such as electrospun fibers (EFs) or nanoparticles (NPs) have been used in a variety of applications like wound dressing, tissue regeneration, and antimicrobial delivery to incorporate bioactive agents such as proteins, peptides, nucleic acids and hydrophilic/hydrophobic drugs. Polymers (e.g., fibers or particles) can protect encapsulated cargo from premature degradation, in addition to minimizing systemic absorption and associated side effects. In addition, electrospun fibers and nanoparticles offer cost-effective, reproducible, and highly tunable methods to provide efficient encapsulation and release based on the needs of rapid-onset or prolonged delivery applications. Many studies have shown that fibers composed of natural, synthetic, and semi-synthetic polymers and polymer blends can tune drug miscibility and that the resulting drug-polymer interactions may lead to different release profiles.

We sought to direct PLGA EFs and NPs to niches of the oral cavity harboring S. gordonii. Co-aggregation factor A (CafA), a fimbrial protein expressed by Actinomyces oris, which constitutes the tip fimbrillin of the type 2 fimbriae of A. oris (see, e.g., FIG. 11B), was used as a targeting ligand to functionalize BAR-encapsulated EFs and NPs. Without being bound by any particular theory, during plaque formation, CafA binds to the GalNAcβ1-3Gal motif of the receptor polysaccharides (RPS) found on certain species of oral commensal streptococci such as S. gordonii and S. oralis, facilitating the co-aggregation of actinomyces and streptococci. Since CafA is a key adhesin that mediates actinomyces-streptococci binding, functionalization of EFs and NPs with CafA should enable actively targeting to areas of the oral cavity harboring commensal streptococci, to gradually release BAR into P. gingivalis-preferred niches (FIG. 11C). Modifying the surface of BAR-encapsulated EFs and NPs with CafA should enhance efficacy by augmenting their adhesion to commensal streptococci, facilitating enhanced retention for durations relevant to oral delivery (˜8 to 12 hr), promoting more gradual release of BAR peptide, and resulting in potent inhibition of P. gingivalis adhesion to S. gordonii in a dual-species biofilm.

The development of an effective oral delivery system that can release BAR within a time frame desired for twice-daily applications can offer an alternative platform that increases loading, facilitates ease of administration, and provides the potential of enhanced retention in the oral cavity.

Electrospinning is a well-known method of producing fibers that uses electric force to produce charged liquid jets of polymer solutions on the order of a few hundred nanometers or less in diameter. Essentially any soluble polymer with a sufficiently high molecular weight, including branched polymers, can be electrospun. Polymers suitable for use in the compositions and methods described herein include those that, once electrospun, can be deposited in the oral cavity without any adverse effects (e.g., toxicity). Representative hydrophobic and hydrophilic polymers that can be used are described herein, although it would be appreciated that the compositions and methods described herein are not limited to those polymers.

Any number of electrospinning methods can be used to produce the electrospun fibers described herein, including, without limitation, co-axial electrospinning, emulsion electrospinning, or melt electrospinning. A number of different fiber morphologies can be produced (e.g., ribbons, beaded, aligned, porous, core-shell), although the fiber morphology is less important than the BAR polypeptide release profile and the suitability of the fiber for delivery to the oral cavity.

Nanoparticles are small particles that generally range in size from about 1 to about 100 nanometers in diameter. The definition given by the European Commission states that at least half of the particles in a sample must be 100 nm or less in diameter. Nanoparticles can occur in a variety of shapes, for which a number of descriptors have been applied (e.g., nanospheres, nanorods, nanochains, nanofibers, and nanoboxes). The shape of nanoparticles can be determined by the intrinsic properties of the material and/or the methods by which they're made. See, for example, Murthy, 2007, Int. J. Nanomedicine, 2:129-41. Similar to the electrospun fibers, however, the shape of the nanoparticles is less important than the BAR polypeptide release profile and the suitability of the nanoparticle for delivery to the oral cavity.

Any number of polymers or combinations of polymers can be used to make nanoparticles. As already discussed herein, polymers suitable for use in the compositions and methods described herein include those that can be deposited in the oral cavity without any adverse effects (e.g., toxicity). Representative hydrophobic and hydrophilic polymers that can be used to produce nanoparticles are described herein, although it would be appreciated that the compositions and methods described herein are not limited to only those polymers.

The BAR polypeptide (SEQ ID NO:1) is known in the art, and originates from the SspB (Antigen I/II) protein sequence of S. gordonii.

(SEQ ID NO: 1)
LEAAPKKVQDLLKKANITVKGAFQLFS

In addition, a BAR polypeptide containing the following substitutions, relative to the double-underlined portion of the BAR polypeptide shown above, would be expected to function similarly as the native BAR polypeptide.

| | | | | | |
|---|---|---|---|---|---|
| Native sequence | N | H | E | V | \|K |

| | | |
|---|---|---|
| Substitutions promoting | R | I |
| P. gingivalis adherence | K | F |
| | H | W |
| | S | |

The SspB (Antigen I/II) from S. gordonii is shown in SEQ ID NO:4 (see, for example, GenBank Accession No. WP_061601630.1). A nucleic acid sequence encoding the SspB (Antigen I/II) is shown in SEQ ID NO:5.

(SEQ ID NO: 4)
MNKRKEVFGF RKSKVAKTLC GAVLGAALIA IADQQVLADE VTETNSTANV AVTTTGNPAT
NLPEAQGEAT EAASQSQAQA GSKDGALPVE VSADDLNKAV TDAKAAGVNV VQDQTSDKGT
ATTAAENAQK QAEIKSDYAK QAEEIKKTTE AYKKEVEAHQ AETDKINAEN KAAEDKYQED
LKAHQAEVEK INNANATAKA EYEAKLAQYQ KDLAAVQKAN EDSQLDYQNK LSAYQAELAR
VQKANAEAKE AYEKAVKENT AKNAALQAEN EAIKQRNETA KANYDAAMKQ YEADLAAIKK
AKEDNDADYQ AKLAAYQAEL ARVQKANADA KAAYEKAVEE NTAKNTAIQA ENEAIKQRNA
AAKATYEAAL KQYEADLAAV KKANEDSEAD YQAKLAEYQT ELARVQKANA DAKAAYEKAV
EDNKAKNAAL QAENEEIKQR NAAAKTDYEA KLAKYEADLA KYKKELAEYP TKLKAYEDEQ
AKIKAALVEL EKNKDQDGYL SKPSAQSLVY DSEPNAQLSL VTEGSFLKAT AVDNAFKQDA
AQYGKKNLQL DNLNVTYLEG AAATSTSMEL YGNINDKSDW TTNVGNKTEV KWGSVLLERG
QSVTATYTNL QNSYYNGKKI SKIVYKYTVD PSSQFKNPSG KVWLGIFTDP TLGVFASAYT
GQVEKDTSLF IKNEFTFYDE NDQPINFDNA LLSVASLNRE HNSIEMAKDY SGTFIKISGS
SIGEKNGMIY ATKTLNFRKD QGGSRWTMYP RANEPGSGWD SSDAPNSWYG AGAISMSGPT
NHVTVGATSA TNVMSVAEMP QVPGRDNTEG KRPNIWYSLN GKIRAVNVPK ITKEKPTPPV
APTEPQAPTY EVEKPLEPAP VAPTYENEPT PPVKTPDQPE PSKPEEPTYE TEKPLEPAPV
VPTYENEPTP PVKTPDQPEP SKPEEPTYET EKPLEPAPVA PTYENEPTPP VKTPDQPEPS
KPEEPTYDPL PTPPLAPTPK QLPTPPVVPT VHFHYSNLLA QPQINKEIKN EDGVDIDRTL
VAKQSIVKFE LKTEALTAGR PKTTSFVLVD PLPTGYKFDL DATKAASTGF DTTYDEASHT
VTFKATDETL ATYNADLTKP VETLHPTVVG RVLNDGATYT NNFTLTVNDA YGIKSNVVRV
TTPGKPNDPD NPNNNYIKPT KVNKNKEGLN IDGKEVLAGS TNYYELTWDL DQYKGDKSSK
EAIQNGFYYV DDYPEEALDV RPDLVKVADE KGNQVSGVSV QQYDSLEAAP KKVQDLLKKA
NITVKGAFQL FSADNPEEFY KQYVATGTSL VITDPMTVKS EFGKTGGKYE NKAYQIDFGN
GYATEVVVNN VPKITPKKDV TVSLDPTSEN LDGQTVQLYQ TFNYRLIGGL IPQNHSEELE
DYSFVDDYDQ AGDQYTGNYK TFSSLNLTMK DGSVIKAGTD LTSQTTAETD ATNGIVTVRF
KEDFLQKISL DSPFQAETYL QMRRIAIGTF ENTYVNTVNK VAYASNTVRT TTPIPRTPDK
PTPIPTPKPK DPDKPETPKE PKVPSPKVED PSAPIPVSVG KELTTLPKTG TNDATYMPYL
GLAALVGFLG LGLAKRKED
(SEQ ID NO: 5)
tt aatcttcttt gogttttgcc agacctaatc ctaggaaacc aacaagggca gccaagccta
agtaaggcat ataagtcgca tcatttgttc ctgtttttgg aagtgttgtt aattctttac
caacagaaac aggaattggt gotgaaggat cttcaacttt tggactagga acttttggtt
cttttggtgt ctcaggtttg tcaggatcct ttggttttgg cgttggaatt ggtgtcggtt
tgtctggtgt tettggtatt ggagttgttg tacgtactgt gttagatgca taagcaacct
tattaacagt atttacataa gtattttcaa atgttccaat agcaattctg cgcatttgaa
ggtaggtttc agcttggaat ggcgaatcca aactaatctt ttgtaaaaag tcttccttga
aacgaacagt tacaatacca tttgtagcat ctgtttcagc agtagtttga gatgttagat
ctgtacctgc cttaatcact gaaccatctt tcattgtcaa gttcagagag ctgaatgtct
tgtaattacc agtatactga tcaccagctt ggtcataatc atccacaaag ctgtaatctt
ctaattcctc agaatgattt tgtggaatga ggccaccaat cagacgatag ttaaatgttt
gatacaattg aactgtttga ccatccagat tttcactagt tggatctagg ctcactgtta
cgtctttttt aggggtgatt ttcggtacgt tgttaaccac tacttctgtc gcataaccat
ttccgaaatc aatttgatag gccttatttt catacttacc acctgtctta ccaaattcag
acttaacagt catcggatct gtaatgacta gtgatgttcc agttgctaca tattgcttgt
aaaattcttc tcgattatca gcagagaaga gttggaaagc acccttaaca gtaatgttag
ctttcttcaa caagtcttga actttcttag gagcggcttc tagactgtca tattgttgaa
cgctgacacc tgatacttga tttccttttt catctgcaac cttaaccaaa tcagggcgaa
catctaaagc ttcttctgga taatcatcta catagtagaa accgttttgg attgcttcct
tagaagattt atcgccctta tattgatcca aatcccatgt taattcgtag tagttggttg
aaccagctaa aacttctttt ccgtcaatat taagaccttc tttattctta tttactttcg
ttggcttaat atagttgtta tttggattat caggatcatt tggtttacca ggagtagtta
cacgaacaac atttgactta atgccataag catcattgac tgtcaaagta aagttattag
tataagttgc tccatcattc aatactcgac caacaaccgt tggatgaaga gtctcaacag
gtttagttaa gtcagcattg tatgttgcca atgtctcatc agtcgcctta aaggttacag
tatgactggc ttcatcataa gttgtatcaa agcctgtact tgcagcctta gttgcatcca
agtcaaactt atagccagtt ggaagcggat ctaccaatac aaatgaagta gtttttggac
gtccagctgt caaagcttca gttttcaatt caaacttgac aattgactgt ttagcaacca
atgtccggtc aatatctaca ccatcctcat tcttgatttc tttattaatc tgaggctgag
ctaataaatt gctatagtgg aaatgaactg tcggtacaac tggtggagtt ggtaactgct
taggagttgg tgctagcggt ggagttggca atggatcata tgttggctct tccggttttg
atggctccgg ttgatctgga gtcttaactg gtggagttgg ctcattttca tatgttggtg
ctactggtgc tggctccaat ggcttctctg tctcatatgt tggctcttcc ggttttgacg
gctccggttg atctggagtc ttaactggcg gagttggctc attttcatat gtcggtacta
ctggtgctgg ctccaatggt ttctctgtct catatgttgg ctcttccggt tttgacggct
ccggttggtc tggagtctta actggtggag ttggctcatt ttcatatgtc ggtgctaccg
gtgctggctc caatggtttt tccacttcat aagtaggagc ttgtggttca gttggtgcta
ccggcggagt tcgtttttcc ttagtaattt tcggaacgtt aacagcacga atcttaccat
tgagcgagta ccagatgttt ggtcttttac cttcggtatt gtctcttcca ggtacttgag
gcatttctgc tacagacatc acattggtag cagatgttgc accaactgta acgtgattcg
taggaccgga catactaatg gcccctgcac cgtaccaaga gtttggtgca tctgatgaat
cccaacctga accaggttca tttgctctag gatacatagt ccaacgagat ccaccctgat
cttttctaaa attgagggtt ttggtagcat aaatcatgcc atttttttca ccgatggatg
aacctgagat tttgataaaa gtaccactat aatccttggc catctcaata gagttatgct
cacggttaag tgaggctaca gaaagaaggg ccttgtcaaa attaattggc tgatcatttt
catcatagaa ggtgaattca tttttaatga aaagcgaagt atctttttct acttgaccag
tataggctga agcaaagact cctagagtag ggtcggtaaa gattcccaac caaacttttc
cactaggatt tttaaattgc gaagaaggat ctacagtata tttgtaaaca atcttcgaaa
tctttttacc attgtagtag gaattttgaa ggttggtata agtcgctgtt acgctttgac
cacgttctag aagaacagat ccccatttaa cttcagtttt attccctaca ttggtggtcc
agtcagattt gtcgttgatg ttaccataca attccataga agtgctagtt gcagctgctc
cttctaaata agtaacgttt agattatcta actgaagatt ctttttacca tattgcgctg
aagatagttg agcatttggt tctgagtcat acactaagct ttgagcagat ggtttagaca
agtatccatc ttgatcttta ttcttttcaa gttctacaag tgccgcctta attttggcct
gctcatcttc ataagccttc aacttagtcg gatactcagc caactctttc ttatatttgg
caagatctgc ttcatattta gctaatttgg cttcatagtc agttttagct gcggcattac
gttgcttgat ttcttcgttt tcagcttgga gcgctgcatt ttttgccttg ttgtcttcaa
cggccttctc ataagctgct ttagcatccg catttgcctt ttgaacacga gccaattccg
tctgatattc ggctagctta gcttgatagt ctgcttcact gtcttcgttg gctttcttta
ctgctgccaa atctgcttca tattgcttca atgctgcttc ataagtcgct ttagctgcgg
cattccgttg cttaatggct tcattttcag cttgaatggc tgtattttta gctgtgtttt
cttcaacagc tttttcatag gctgctttag catcagcatt tgccttttga acacgtgcca
actccgcttg gtaagctgct aattttgctt ggtagtcagc atcattatct tccttagctt
ttttaatagc tgcaagatct gcttcatact gcttcatcgc tgcgtcatag tttgctttgg
cagtttcatt gcgttgctta atcgcttcat tttcagcttg gagtgcagca tttttagcag
tattttcctt tactgctttt tcataagctt ctttagcttc agcattcgct ttttgaacac
gagccaattc tgcttggtaa gctgaaagct tattttgata atcaagctga ctgtcttcat
tagctttttg aacagcggct aaatcttttt gatattgagc tagcttcgct tcatattctg
cctttgctgt cgcattagca ttgttgattt tttcaacttc agcttgatga gctttcaaat
cttcttgata cttatcttca gcagctttgt tttcagcatt gattttatct gtttcagcct
gatgagcctc tacttctttt ttataagctt cagttgtttt ctttatttct tctgcttgtt
tagcgtaatc actcttgatt tcagcttgct tttgcgcatt ttctgcggca gttgtggctg
tacctttatc gcttgtttga tcttgtacaa cattgacacc tgctgctttg gcatccgtca
ctgctttatt taaatcatca gcagatactt ctactggcag agcaccatct ttagaaccag
cctgtgcttg actttggctt gctgcttcag tcgcttctcc ctgtgcttca ggcagattag
tagctggatt tccagttgta gtgactgcta catttgctgt actatttgtt tctgtaactt
catcagccaa aacttgctga tctgcaatag caatcaaagc cgcccctaag acagctccac
ataaagtctt agcgacttta cttttacgaa aaccaaaaac ttcttttctt ttgttcat

The CafA polypeptide used herein is shown in SEQ ID NO:6. In addition to CafA, another protein that adheres specifically to a species of streptococci (e.g., S. gordonii, S. sanguis, S. oralis) to which P. gingivalis adheres will function similarly. For example, CafA-related proteins from other Actinomyces species can be used to target an EF or a NP, or surface proteins from other streptococci that adhere to a species of streptococci (e.g., S. gordonii, S. sanguis, S. oralis) to which P. gingivalis adheres will function similarly.

(SEQ ID NO: 6)
MLRRFFVRSHRRELTSSRQPSRQSRRRLRSGAAISTFALIAGALGTATL
PAPPAEAVHGNPAGGSGKYTQVIDWIDWTEMTNTLPGKGTRILPDGSTG
VVWSTPSRISGDKWRTSRCTLSNVRTTAVGKNETGLPTDRGLSVGYYPG
NWRGDGLARLYNDGTNYTAGVAKKPYATSSNLPLGIANLRDSSTQQFQI
ECSAYLVTSASQPAKSQLEHFPNKVEVPMEGIVFADAEASSWHIPNGQK
EYISVSPIPYVASQNVTYRLLESARPPGCITNSVVGKVSVSTPVGNRPG
VKLRPDDAECSAKVPNGYGPSSVMLLSNMRTGYVEIHGGGRGAVAFGVV
SYMDYGDAPESYGVAGSAFQPAWSGGELSDSGERNIARGKAPYEGDAGD
WYNLSAATDQGNVATTNSAVVRLGALTDHDDTITHSADASRDNTTDIDD
EDALPAGWDRVIWTDIGQKWSQEITCSGSNTKIAGWVDWNRDGTFSAGE
RSAVTSCSSAGKATVTWTVPQDAKRSTINGNGAATFMRLRITGAPPNGR
EAEDPQPTGIAVNGEVEDHQVQVQLPNLSLAKQVDNTAAGSLGLSAKDW
TLTATPKRGKTASGAGGFDSAYLPQGQTVLSESSSSPKSAGYKASVSCV
PHPNSDIRTPSSTVNSASKTLDLATGEWMQCTMVNTAQPGQVVWSKVDD
AGNPLAGTVFTLASPALNGGQKEVTDCVVTGGKATCPNGSVDQDPRAGF
FRVSGLTWGNYSITETQAPAGYHLSSTTLTKTLDGSAPAAGTDDDTPTL
DLGQVTNTRIKGSATWTKTDERGNPIKGAQWSLVPLDSNGRPQPDQART
ITDCVGTCAQGSLDTDGNPGAFKLAELGYGSYGLMETKPPTGYILDATP
RTITISSQGQVVALGKISNRKSAVPAIPFTGGSAADTFLIAGGAVLGIT
APVMMIQAYRRRRALDS

In addition to the nucleic acid (e.g., SEQ ID NO: 5) and polypeptides (e.g., SEQ ID NOs: 1, 4 and 6) disclosed herein, the skilled artisan will further appreciate that changes can be introduced into a nucleic acid molecule, thereby leading to changes in the amino acid sequence of the encoded polypeptide. For example, changes can be introduced into nucleic acid coding sequences using mutagenesis (e.g., site-directed mutagenesis, PCR-mediated mutagenesis) or by chemically synthesizing a nucleic acid molecule having such changes. Such nucleic acid changes can lead to conservative and/or non-conservative amino acid substitutions at one or more amino acid residues. A “conservative amino acid substitution” is one in which one amino acid residue is replaced with a different amino acid residue having a similar side chain (see, for example, Dayhoff et al. (1978, in Atlas of Protein Sequence and Structure, 5(Suppl. 3):345-352), which provides frequency tables for amino acid substitutions), and a non-conservative substitution is one in which an amino acid residue is replaced with an amino acid residue that does not have a similar side chain.

Nucleic acids and polypeptides that differ from SEQ ID NO: 5 and SEQ ID NOs: 1, 4 and 6, respectively, also are provided. Nucleic acids and polypeptides that differ in sequence from such sequences can have at least 80% sequence identity (e.g., at least 85%, 90%, 95%, or 99% sequence identity) to SEQ ID NO: 5 or SEQ ID NOs: 1, 4 or 6, respectively.

In calculating percent sequence identity, two sequences are aligned and the number of identical matches of nucleotides or amino acid residues between the two sequences is determined. The number of identical matches is divided by the length of the aligned region (i.e., the number of aligned nucleotides or amino acid residues) and multiplied by 100 to arrive at a percent sequence identity value. It will be appreciated that the length of the aligned region can be a portion of one or both sequences up to the full-length size of the shortest sequence. It also will be appreciated that a single sequence can align with more than one other sequence and hence, can have different percent sequence identity values over each aligned region.

The alignment of two or more sequences to determine percent sequence identity can be performed using the algorithm described by Altschul et al. (1997, Nucleic Acids Res., 25:3389 3402) as incorporated into BLAST (Basic Local Alignment Search Tool) programs, available at ncbi.nlm.nih.gov on the World Wide Web. BLASTN is the program used to align and compare the identity between nucleic acid sequences, while BLASTP is the program used to align and compare the identity between amino acid sequences. When utilizing BLAST programs to calculate the percent identity between a sequence and another sequence, the default parameters of the respective programs generally are used.

Nucleic acid fragments are included in the invention. Nucleic acid fragments suitable for use in the invention are those fragments that encode a polypeptide having functional activity. These fragments can be called “functional fragments,” although it is understood that it is not the nucleic acid that possesses functionality.

In accordance with the present invention, there may be employed conventional molecular biology, microbiology, biochemical, and recombinant DNA techniques within the skill of the art. Such techniques are explained fully in the literature. The invention will be further described in the following examples, which do not limit the scope of the methods and compositions of matter described in the claims.

### EXAMPLES

Part A—Rapid Release Polymeric Fibers for Inhibition of Porphyromonas gingivalis and Streptococcus gordonii Biofilms

### Example 1—Materials

Hydrophobic polymers including poly(lactic-co-glycolic acid) (PLGA, 50:50 lactic:glycolic acid, MW 30,000-60,000), poly(L-lactic acid) (PLLA, MW 50,000), and polycaprolactone (PCL, MW 80,000), and the hydrophilic polymer, polyethylene oxide (PEO, MW 100,000) were purchased from Sigma-Aldrich (St. Louis, MO, USA). Tris-EDTA (TE) buffer (pH 8.0), phosphate buffered saline (PBS), and the organic solvents chloroform, dimethyl sulfoxide (DMSO), and hexafluoroisopropanol (HFIP) were also purchased from Sigma-Aldrich (St. Louis, MO, USA). All chemicals were used directly without further purification. One milliliter plastic syringes, petri dishes, and 20 mL scintillation vials were obtained from VWR. One milliliter glass syringes were purchased from Fisher Scientific. The electrospinner was provided courtesy of Dr. Stuart Williams at the Cardiovascular Innovative Institute, University of Louisville.

### Example 2—Peptide Synthesis

The peptide used in this study (NH₂-LEA APK KVQ DLL KKA NIT VKG AFQ LFS-COOH) (SEQ ID NO: 1) (Daep et al., 2008, Infect. Immun. 76:3273-80) was synthesized by BioSynthesis, Inc. (Lewisville, TX). It was obtained with purity greater than 94% and comprised residues 1167 to 1193 of the SspB (Antigen I/II) protein sequence of S. gordonii. A fluorescent BAR (“F-BAR”) peptide, synthesized by covalently attaching 6-carboxyfluorescein (F-BAR) to the epsilon amine of the lysine residue underlined in the sequence above, was used to more easily characterize BAR loading and release from the fibers via fluorescence detection.

### Example 3—Preparation of Polymer Solutions

To prepare the hydrophobic-only (non-blended) polymer fiber batches, PLGA and PLLA were dissolved in HFIP at a concentration of 15% (w/w), while PCL was dissolved in HFIP at a concentration of 12% w/w due to increased viscosity. The polymer solutions were aspirated into a 7 mL glass scintillation vials, and sealed using aluminum foil and parafilm to prevent evaporation of the organic solvent. The vials were placed in a shaker at 150 rpm and incubated at 37° C. overnight to solubilize the polymer. The final volume of each polymer solution was 1 mL. The following day, the F-BAR peptide was dissolved in 200 μL TE buffer and mixed with the polymer solvents at a concentration of 1% w/w (e.g., 2.4 mg F-BAR/240 mg polymer).

To prepare blended polymers, the hydrophobic polymers PLGA, PLLA, and PCL were mixed with PEO at different ratios (40:60, 20:80, 10:90 w/w) to form PLGA:PEO, PLLA:PEO, and PCL:PEO blends in chloroform at a concentration of 15% (w/v). The blended solutions were aspirated into 20 mL glass scintillation vials, and sealed using parafilm to prevent evaporation of the organic solvent. The vials were placed in a shaker at 150 rpm and incubated at 37° C. overnight to solubilize the polymer. The final volume of each polymer solution was 1 mL.

The following day, the F-BAR peptide was dissolved in 60 μL DMSO. The F-BAR solutions were mixed with the polymer solvent at a concentration of 1% w/w (BAR/polymer content).

### Example 4—Electrospinning

For the non-blended polymer solutions, 1 mL of the mixed polymer suspension was aspirated into a 1 mL plastic syringe with an 18-gauge blunt needle tip. The internal diameter of the BD plastic syringe (4.78 mm), was set in the syringe pump program. The collector was adjusted such that there was at least 10 cm distance maintained from the needle tip. The syringe pump motor controls were adjusted by setting the “slide” control to 4.5 and the “rotor” to 8. The voltage supply was set at 20 kV, and the syringe pump flow rate was set to 0.8 mL per hour. The polymer solution was electrospun at room temperature, under atmospheric conditions, for 1 hr 15 min, and the resulting fine mist was collected on the mandrel and allowed to dry for 15 min. The mandrel was removed from the collector and the fiber was cut and gently peeled off the mandrel. The fiber was placed in a labeled petri dish and kept in a desiccator for 24 hr before characterization. The desiccated fibers were stored in 4° C. until use.

For the blended polymer solutions, 1 mL of the mixed dual-polymer suspension was aspirated into a 1 mL glass syringe with a 22-gauge blunt needle tip. The internal diameter of the Hamilton gastight syringe (4.61 mm), was set in the syringe pump program. A distance of 15 cm was kept between the needle tip and the collector. The “slide” control was set to 4.5 and the “rotor” control was set to 8. A voltage of 20-25 kV was applied, at a flow rate of 0.3 mL per hr. The electrospinning processes were employed under ambient conditions for 3 hr 20 min. The stretched and solidified polymeric fibers were collected on a 4 mm diameter stainless steel mandrel and allowed to dry for 15 min. Similar desiccation and storage conditions were followed, as noted for the non-blended fibers.

### Example 5—EF Characterization: EF Morphology, Diameter, BAR Loading, and Release

Fiber morphology and size were evaluated using scanning electron microscopy (SEM) (JSM-820, JEOL, Tokyo, Japan), and fiber diameters were obtained by analyzing SEM images with NIH ImageJ. The loading and encapsulation efficiency (EE) of F-BAR peptide in the non-blended and blended fibers were determined by dissolving F-BAR fibers in DMSO. The fiber solution was subsequently vortexed, sonicated for 5 min, and dissolved for 1 hr in a dark room. The quantity of extracted F-BAR was determined by measuring the fluorescence using a spectrophotometer (488/518 nm excitation/emission), relative to an F-BAR standard. A standard curve of F-BAR was obtained by adding 0.1 mg F-BAR to 1 mL of 1:9 DMSO:TE, and serially diluting in 1:9 DMSO:TE. The diluted solutions (100 μL/well) were transferred to a 96-well clear bottom microtiter plate in triplicate. For the dissolved fiber samples, after the incubation period, the fiber sample solutions were vortexed and sonicated again. The solutions were diluted 1:2, 1:5, 1:10 and 1:100 in 1:9 DMSO:TE solution, and transferred to a microtiter plate.

The in vitro release of F-BAR from fibers was measured by gentle agitation of EFs in phosphate buffered saline (PBS, pH 7.4) at 37° C. At fixed time points (1, 2, 4, 8, 12, and 24 hr), samples were collected and the amount of F-BAR released from the EFs was quantified via fluorescence spectroscopy, against an F-BAR standard in PBS.

### Example 6—Growth of Bacterial Strains

P. gingivalis (ATCC 33277) was grown in Trypticase soy broth (Difco Laboratories Inc., Livonia, MI, USA) supplemented with 0.5% (w/v) yeast extract, 1 μg/mL menadione, and 5 μg/mL hemin. The medium was reduced for 24 hr under anaerobic conditions (10% CO₂, 10% H₂, and 80% N₂) and P. gingivalis was subsequently inoculated and grown anaerobically for 48 hr at 37° C. S. gordonii DL-1 was cultured aerobically without shaking in brain-heart infusion broth (Difco Laboratories Inc.) supplemented with 1% yeast extract for 16 hr at 37° C.

### Example 7—Biofilm Inhibition Assay

To assess the effectiveness of BAR-incorporated EFs to prevent the interaction of P. gingivalis with S. gordonii, S. gordonii was harvested from culture and labeled with 20 μL of 5 mg/mL hexidium iodide for 15 min at room temperature. Following incubation, cells were centrifuged to remove unbound fluorescent dye. The bacterial concentration was subsequently measured by the OD (600 nm) from twenty-fold diluted cultures of S. gordonii. The optical density of S. gordonii cells was adjusted to 0.8 OD (1×10⁹ CFU/mL) to obtain uniformity between cell counts in each well. After adjusting the optical density, 1 mL of S. gordonii cells was added to each well of 12-well culture plates containing a sterilized micro-coverslip. The cell culture plates were wrapped in aluminum foil to protect the labeled cells from light and placed on a rocker platform in the anaerobic chamber for 24 hr.

P. gingivalis cultures were optimized using a similar approach, utilizing a different fluorescent label (20 μL of 4 mg/mL carboxyfluorescein-succinylester). P. gingivalis was incubated with the fluorescent dye for 30 min on a rocker platform and protected from light. The same procedures were followed as performed with S. gordonii to determine cell concentration, with slight adaptations. The optical density of P. gingivalis was adjusted from 0.8 to 0.4 OD (5×10⁷ CFU/mL) by diluting P. gingivalis cultures with an equal volume of 1×PBS containing BAR-EFs, free BAR, or blank EFs as a control, to a final volume 1 mL. The final concentration of BAR-EFs or free BAR ranged from 0.3-3 μM based on the previously determined IC50 of free BAR (1.3 μM). P. gingivalis was incubated with BAR-EFs, free BAR, or blank EFs at 25° C. for 30 min before transferring to wells containing S. gordonii.

Plates containing P. gingivalis and S. gordonii were subsequently incubated for 24 hr at 37° C. in anaerobic conditions. The following day, the supernatant was removed and cells were washed with PBS. Adherent cells were fixed with 4% (w/v) paraformaldehyde and the cover glass was mounted on a glass slide. Biofilms were visualized using a Leica SP8 confocal microscope (Leica Microsystems Inc., Buffalo Grove, IL) under 60× magnification. Background noise was minimized using software provided with the Leica SP8 and three-dimensional z-stack biofilm images were obtained from 30 randomly chosen frames using a z-step size of 0.7 μm. Images were analyzed with Volocity image analysis software (version 6.3; Perkin Elmer, Waltham, MA, USA) to determine the ratio of green to red fluorescence (GR), representing P. gingivalis and S. gordonii, respectively. Control samples were used to subtract background levels of auto-fluorescence.

Briefly, triplicate samples of S. gordonii alone were immobilized without P. gingivalis or BAR in 12-well culture plates and the same procedures for dual-species biofilm were followed. S. gordonii-only coverslips were visualized and images were analyzed as described above. The GR background was subtracted using the following formula: GR sample or control—GR S. gordonii-only.

Each treatment group (BAR-EFs or free BAR) was analyzed in triplicate and three independent frames were measured for each well. GraphPad InStat (La Jolla, CA) was used for data analysis and differences were considered to be statistically significant when P≤0.05. The percent inhibition of P. gingivalis adherence was calculated with the following formula: GR sample/GR control.

### Example 8—Biofilm Disruption Assay

The same procedures utilized in the inhibition assay were followed, except P. gingivalis was allowed to adhere to streptococci in the absence of BAR peptide or BAR-EFs to demonstrate the ability of BAR-incorporated EFs to disrupt or “treat” pre-established biofilms. The resulting P. gingivalis S. gordonii biofilms were then treated for the maximum duration observed for free BAR to disrupt existing biofilms (3 hr). Established biofilms were administered BAR-EFs, free BAR or blank EFs at various concentrations in 1 mL PBS, and processed and analyzed as described above. The mean and standard deviation (SD) between samples were determined and the percent disruption of P. gingivalis adherence was calculated with the following formula: GR sample/GR control.

### Example 9—Tissue Culture

Telomerase immortalized gingival keratinocytes (TIGKs) were grown on 12-well collagen-coated plates (Becton Dickinson, Bedford, MA) and cultured using DermaLife K Calcium Free Medium (LifeFactors®) supplemented with penicillin/streptomycin (100 U/mL final concentration; St. Louis, MO), insulin (5 μg/mL), recombinant human (rh), L-glutamine (6 mM), apo-transferrin (5 μg/mL), epinephrine (1 μM), rh TGF-α (0.5 ng/mL), extract P™, calcium chloride (0.06 mM) and hydrocortisone hemisuccinate (100 ng/mL). The cells were incubated at 37° C. in the presence of 5% CO₂ for 6 days until they reached 95% confluence.

### Example 10—Determination of BAR and BAR-EFs Safety In Vitro

Hemolytic Assay: A sample of 250 μL of 1% sheep erythrocytes (Rockland Inc, Pennsylvania, USA) was suspended in sterile PBS. The IC50 and maximum effective concentrations (1.3 and 3.4 μM, respectively) of free BAR peptide or BAR in 10:90 PLGA:PEO BAR-EFs used in in vitro and in vivo studies, were added to sheep erythrocytes. Water replaced PBS as a positive control for cell hemolysis. The suspension was incubated at 37° C. for 3 hr then centrifuged at 3,500×g. Hemoglobin released due to cell lysis was analyzed by measuring the absorbance at 541 nm.

MTT Assay: TIGK cells were seeded in 12-well plates at a density of 6×10⁴ cells in 1 mL media per well and incubated for 24 hr to allow for 60-70% confluency and sufficient adhesion. Cells were treated with 1.3 or 3.4 μM of BAR or 10:90 PLGA-PEO BAR-EFs. After 24 hr, 100 μL of MTT solution was added to the media of all samples. After 4 hr incubation at 37° C., 550 μL of lysis buffer was added to the media of each well and plates were incubated for overnight. The absorbance of each well was read at 570 nm, and the sample absorbance was normalized to the absorbance of medium-only treated cells. Cells were treated with 10% DMSO media (100 μL DMSO in 900 μL media) as a positive control for cell death.

ATP Assay: The metabolic activity of TIGK cells was assessed by measuring total ATP levels using the CellTiter-Glo reagent (Promega, Madison WI), as described by the manufacturer. TIGK cells were seeded at a density of 6×10⁴ cells in 1 mL media per well and incubated at 37° C., 5% CO₂ for 24 hr in a 12-well flat bottom plate. Cells were then incubated with BAR or 10:90 PLGA-PEO BAR-EFs (1.3 or 3.4 μM) for 24 hr at 37° C. in 5% CO₂. Cells were then lysed with 500 μL of 0.1% Triton X-100 for 30 min at 37° C. The lysates were collected and centrifuged at 1,000×g for 10 min at 4° C., and 50 μL of supernatant was mixed with 50 μL of CellTiter-Glo reagent. Samples were incubated at ambient temperature for 10 min in a black 96-well plate in the dark. Total luminescence was measured with a Victor 3 luminometer (Perkin-Elmer, Inc). Cells incubated with 1 ng of staurosporine or with medium-only served as positive and negative controls for cell death, respectively.

LDH Assay: Cell membrane leakage was measured by assessing the release of lactate dehydrogenase (LDH). Extracellular LDH was quantified using a CytoTox96® non-radioactive cytotoxicity assay (Promega, Madison WI) as described by the manufacturer. TIGK cells were plated at density of 6×10⁴ cells in 1 mL media per well in a 12-well flat bottom plate, and incubated at 37° C., 5% CO₂ for 24 hr. BAR or 10:90 PLGA-PEO BAR-EFs (1.3 or 3.4 μM) were added to cells in triplicate for 24 hr at 37° C. in 5% CO₂. Fifty microliters of supernatant from free BAR and BAR-EF-treated (1.3 and 3.4 μM) cells were added to the LDH substrate and incubated at room temperature for 30 min. Then the reactions were terminated by adding 50 μL of stop solution. LDH activity was determined by measuring the optical density of the solution at 490 nm. Cells incubated with staurosporine or with medium-only served as positive and negative controls for toxicity, respectively.

Oxidative DNA Damage: Free radicals and other reactive species are generated from cells under stress and cause oxidative damage to biomolecules. DNA is the most targeted site of oxidative attack. The apurinic/apyrimidine (AP or abasic) site is a prevalent oxidative DNA damage lesion. OxiSelect™ Oxidative DNA Damage Quantitation Kit (Cell Biolabs, INC., San Diego, CA, USA) was used to quantify AP sites in cells treated with free BAR or 10:90 PLGA-PEO BAR-EFs (1.3 or 3.4 μM) as described by the manufacturer. TIGK cells were plated at density of 6×10⁴ cells in 1 mL media per well in a 12-well flat bottom plate, and incubated at 37° C., 5% CO₂ for 24 hr. BAR or BAR-EFs (1.3 or 3.4 μM) were added to cells in triplicate for 24 hr at 37° C. in 5% CO₂. Cells treated with 2 mM H₂O₂ or medium-only served as positive and negative controls for DNA damage, respectively. Genomic DNA was isolated from TIGK cells by QIAamp DNA Mini kit (Qiagen). AP sites were determined in genomic DNA by using a biotinylated aldehyde reactive probe (ARP) that reacts specifically with an aldehyde group of AP sites, followed by colorimetric detection using a streptavidin-enzyme conjugate (450 nm). The quantity of AP sites in DNA samples was determined by comparing the absorbance with standard curve of known amount of AP sites.

### Example 11—Statistical Analysis

Data from each of the toxicity tests were analyzed using ANOVA after passing Bartlett's and Brown-Forsythe tests for homogeneity of variances using GraphPad InStat (La Jolla, CA). A pair-wise, parametric analysis of variance using a Bonferroni multiple comparison post hoc test was used to determine the statistical difference among the individual groups. A P-value of ≤0.05 was considered to be statistically significant.

### Example 12—EF Characterization: EF Morphology, Diameter, BAR Loading, and Release

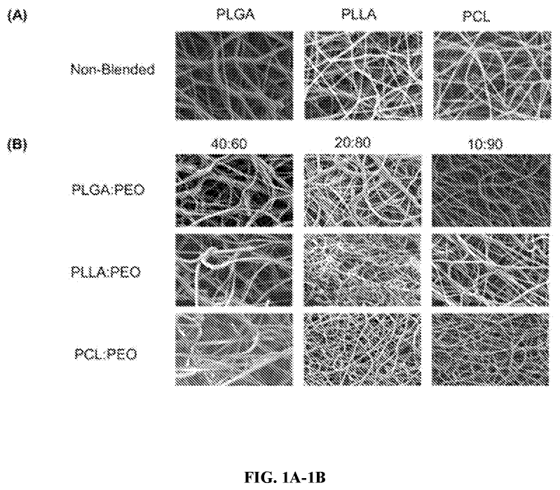

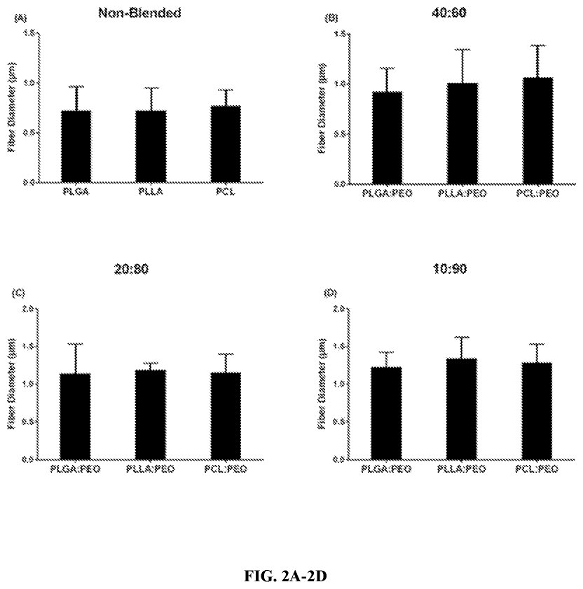

Fiber morphologies and diameters are shown in FIGS. 1 and 2. The average diameters of EFs ranged from 0.7 to 1.3 μm with no statistical significance observed within or across different formulations, as a function of polymer type or blend ratio.

### Example 13—BAR Loading and Release

The overall polymer yield after electrospinning ranged from 40-60% for the non-blended fiber formulations, while the blended fibers achieved higher yields spanning 80-90%. The total F-BAR loading for non-blended and blended EFs ranged between 4.6-6.9 μg BAR/mg polymer and 6.0-9.2 μg BAR/mg polymer, respectively, indicating that high loading of F-BAR was achieved in all fiber formulations (Table 1). High loading and encapsulation efficiency were achieved in all fiber formulations. However, non-blended EFs showed comparatively lower polymer yield and encapsulation efficiency, relative to the blended EFs. Data represent the mean±standard deviation (n=3) of three independent samples.

TABLE 1
The amount of BAR loaded in non-blended and blended
polymeric EF formulations (μg/mg) and percent
of total BAR loaded in blended and blended EFs

| | | Overall | Loading | Encapsulation |
|---|---|---|---|---|
| Fiber | Blend | polymer yield | BAR/fiber | efficiency |
| formulation | ratio | (%) | (ug/mg) | (%) |
| PLGA | 100:0 | 59.0 | 6.9 ± 0.1 | 69 ± 2.5 |
| PCL | | 51.0 | 6.0 ± 0.4 | 60 ± 4.0 |
| PLLA | | 42.3 | 4.6 ± 0.6 | 46 ± 5.2 |
| PLGA:PEO | 40:60 | 82.9 | 7.4 ± 0.5 | 74 ± 5.5 |
| PCL:PEO | | 91.5 | 8.6 ± 0.2 | 86 ± 2.4 |
| PLLA:PEO | | 82.0 | 9.1 ± 0.3 | 92 ± 3.1 |
| PLGA:PEO | 20:80 | 80.9 | 8.8 ± 0.2 | 88 ± 2.6 |
| PCL:PEO | | 89.3 | 8.9 ± 0.4 | 89 ± 4.0 |
| PLLA:PEO | | 85.2 | 8.3 ± 0.4 | 83 ± 4.2 |
| PLGA:PEO | 10:90 | 82.8 | 8.8 ± 0.5 | 88 ± 5.6 |
| PCL:PEO | | 80.0 | 6.0 ± 0.4 | 60 ± 4.0 |
| PLLA:PEO | | 80.9 | 8.5 ± 0.3 | 85 ± 3.5 |

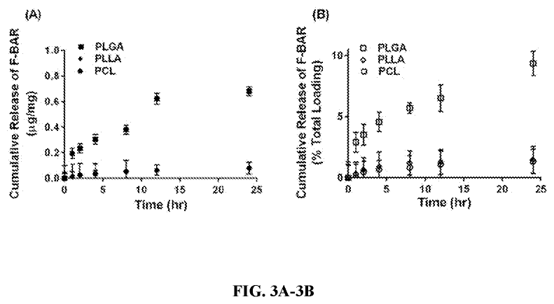

To determine the amount of F-BAR release from the different fiber formulations, F-BAR EFs were incubated in PBS at 37° C. The fluorescence of the collected supernatant was measured at 1, 2, 4, 8, 12 and 24 hr. FIG. 3 shows the cumulative release of F-BAR from non-blended EFs at each time point over a 24 hr duration. PLGA EFs demonstrated minimal release of F-BAR (9.5% of total loading) after 24 hr, while PLLA and PCL fibers showed even less release during the same duration. Overall, EFs consisting of only hydrophobic polymers (i.e., non-blended formulations) demonstrated minimal release relative to the PEO-blended EFs.

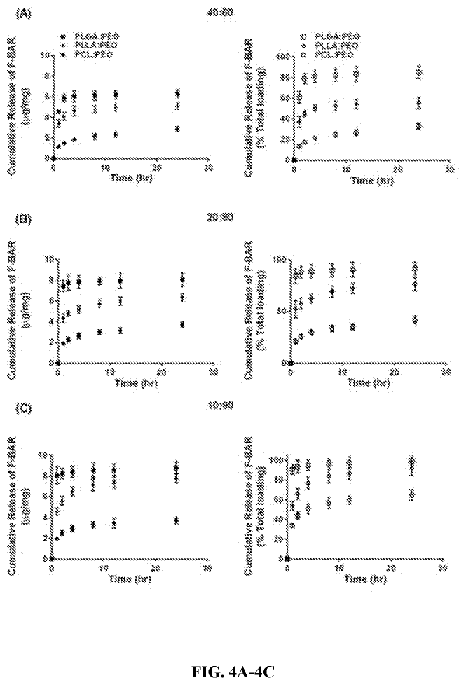

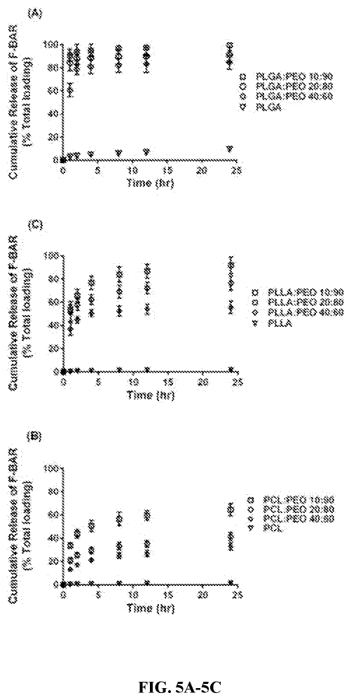

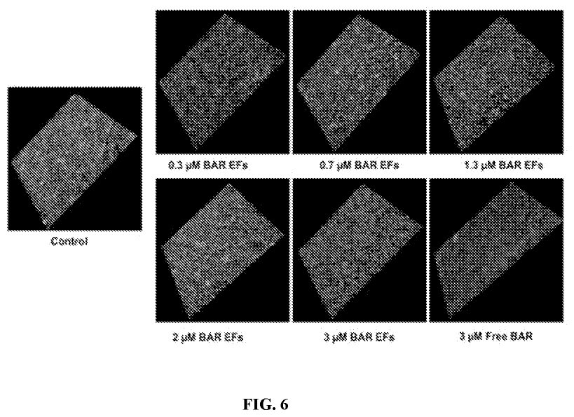

The release of F-BAR from blended PLGA:PEO, PLLA:PEO and PCL:PEO fibers with different blend ratios (40:60, 20:80, 10:90) as a function of hydrophobic polymer type, is shown in FIG. 4. The importance of the PEO ratio in each hydrophobic fiber type, is emphasized in FIG. 4, with the 10:90 formulation providing maximum release of F-BAR for each hydrophobic blend. Fibers comprised of 10:90 PLGA:PEO released 8.39±0.06 μg BAR/mg EF, corresponding to 95±0.26% of the incorporated F-BAR within the first 4 hr, relative to PLLA:PEO and PCL:PEO 10:90 fibers with 76.8±0.8% and 50.6±0.8% of F-BAR release, respectively (FIGS. 4 and 5). A significant reduction in the release of F-BAR was observed after 4 hr for the 10:90 PLGA:PEO EFs and after 8 hr for the other 10:90 PEO-blended formulations (FIG. 4). For the 20:80 blended formulations, the PLGA:PEO fibers showed a maximum release of 88.7±0.3%, compared to PLLA:PEO and PCL:PEO with 62.4±2.1% and 29.6±0.06% release, respectively, after 4 hr. Similar trends in F-BAR release were observed for the 40:60 formulations with PLGA:PEO exhibiting the maximum release of 81.2±0.1%, and PLLA:PEO and PCL:PEO releasing 50.6±3.1% and 21.3±0.2% after 4 hr. Of the tested formulations, 40:60 PLGA:PEO, PLLA:PEO and PCL:PEO released the least F-BAR within the first 4 hr, and a significant reduction in release was observed after ˜4 hr for both the 20:80 and 40:60 formulations. Overall, the release trends for the different ratios of polymer blends were similar, with PLGA blends achieving the highest F-BAR release, followed by PLLA and PCL formulations.

### Example 14—P. gingivalis/S. gordonii Biofilm Inhibition

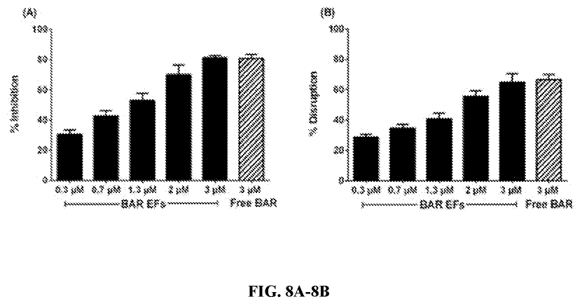

Given that the 10:90 PLGA:PEO blends achieved the highest release of F-BAR, the ability of the 10:90 PLGA:PEO BAR-EFs to inhibit or “prevent” P. gingivalis biofilm formation was assessed, relative to the administration of free BAR. To assess inhibition, 10:90 PLGA:PEO BAR-EFs or free BAR were administered to P. gingivalis for 24 hr. Subsequently, BAR-EF or free BAR-treated P. gingivalis was incubated with immobilized S. gordonii. As shown in FIGS. 6 and 8A, P. gingivalis adherence was significantly reduced in the presence of 10:90 PLGA:PEO BAR-EFs. Biofilm formation was inhibited by 31, 42, or 82% by 0.3, 0.7, and 3.0 μM BAR-EFs, respectively. The maximum inhibition observed was similar to the 81% inhibition observed with free BAR (3 μM). BAR-incorporated EFs potently inhibited biofilm formation in a dose-dependent manner (IC50=1.3 μM). As expected, no statistical significance (P>0.05) in inhibition was observed between BAR-incorporated EFs and free BAR.

### Example 15—P. gingivalis/S. gordonii Biofilm Disruption

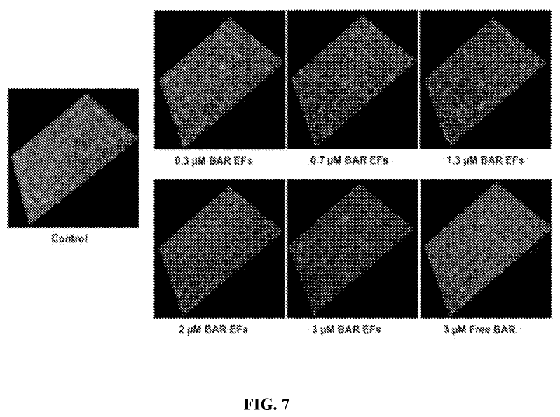

The ability of the 10:90 PLGA:PEO BAR-incorporated EFs to disrupt or “treat” pre-existing P. gingivalis/S. gordonii biofilms was assessed (FIGS. 7 and 8B). Dual-species biofilms were formed for 24 hr, and were subsequently incubated for 3 hr with BAR-incorporated EFs or free BAR. Biofilm formation was disrupted by 29, 34, or 66% by 0.3, 0.7, and 3.0 μM BAR-EFs. The maximum inhibition observed was similar to the 66% inhibition observed with free BAR (3 μM). Taken together, BAR-EFs exhibited effective biofilm disruption (IC50=2 μM) that was similar to free BAR (P>0.05).

### Example 16—Assessment of BAR and BAR-EFs In Vitro Cytotoxicity

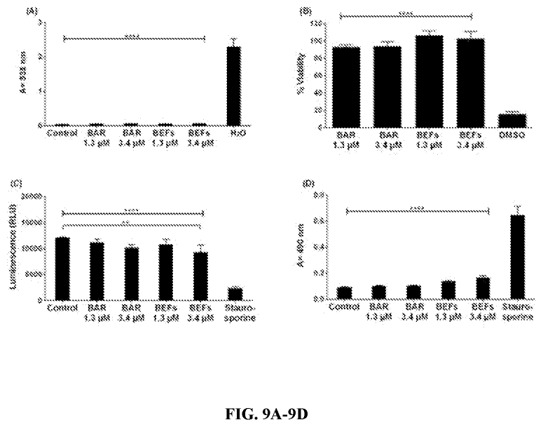

Hemolytic Assay: The cytotoxicity of free BAR and 10:90 PLGA:PEO BAR-EFs was initially assessed by measuring the hemolytic activity against sheep red blood cells (RBCs). As shown in FIG. 9A, neither free BAR nor BAR-EFs (1.3 or 3.4 μM BAR) induced hemolysis of RBCs.

MTT Assay: To determine the effect of free BAR or BAR-EFs on TIGK cell viability, cells were treated with free BAR or 10:90 PLGA:PEO BAR-EFs (1.3 or 3.4 μM) and viability was assessed using the MTT assay. As shown in FIG. 9B, free BAR (1.3 or 3.4 μM) treated cells exhibited no loss in viability (P>0.05), while BAR-EF (1.3 or 3.4 μM) treated cells showed higher viability (P≤0.05), relative to medium-only treated cells.

ATP Assay: The metabolic activity of TIGK cells was assessed by measuring ATP levels. As shown in FIG. 9C, cells treated with free BAR (1.3 or 3.4 μM) or 10:90 PLGA:PEO BAR-EFs (1.3 μM) showed no decrease in ATP relative to medium-only treated cells, while, cells treated with 10:90 PLGA:PEO BAR-EFs (3.4 μM) exhibited slightly lower levels of ATP relative to medium-only treated cells (9303.5±1399 and 12094±181 relative light units (RLUs), respectively, P≤0.01). Staurosporine-treated cells demonstrated significantly lower levels of ATP (P≤0.0001) than were observed for medium-only, free BAR, and 10:90 PLGA:PEO BAR-EF treated cells.

LDH Assay: Since some peptides are known to damage the cell membrane, LDH released in the cell media was evaluated as a marker for cell membrane integrity after free BAR or 10:90 PLGA:PEO BAR-EF treatment. FIG. 9D shows that free BAR or BAR-EFs (1.3 or 3.4 μM) induced no change in levels of LDH released from cells, relative to medium-only treated cells. However, staurosporine induced a significantly higher level of LDH released from TIGK cells relative to cells treated with medium-only, free BAR, and BAR-EFs (P≤0.0001).

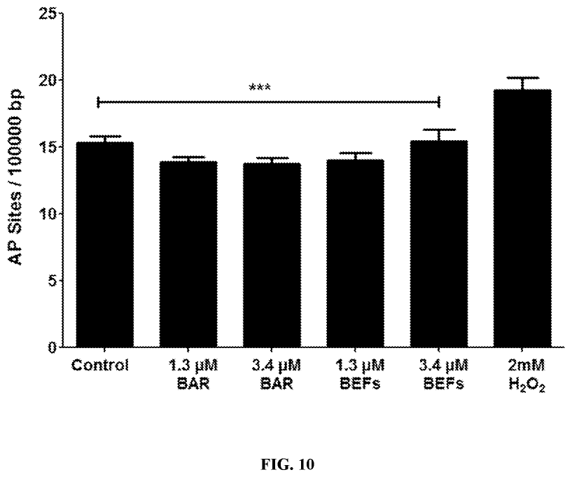

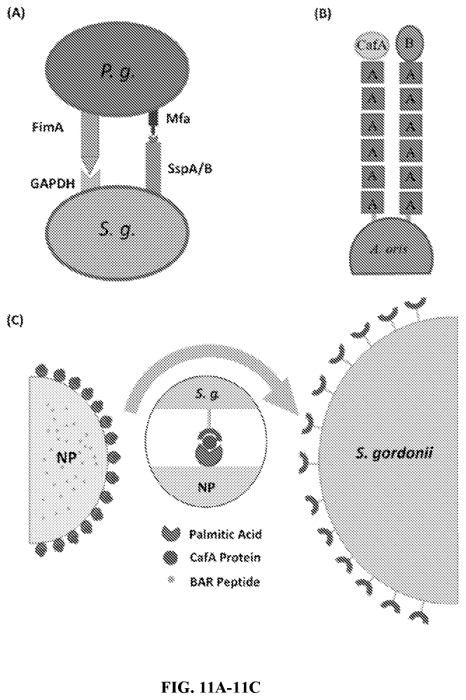

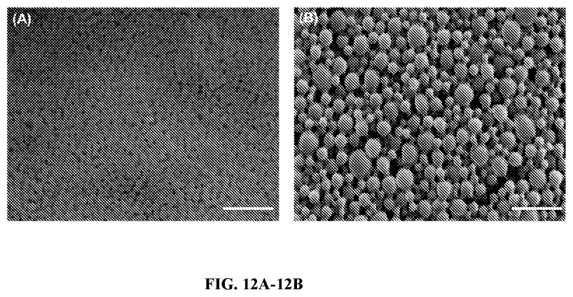

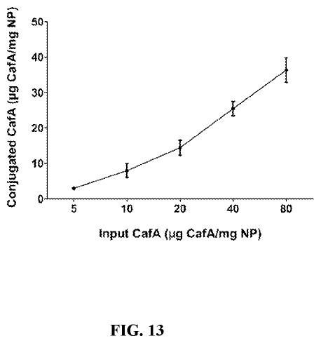

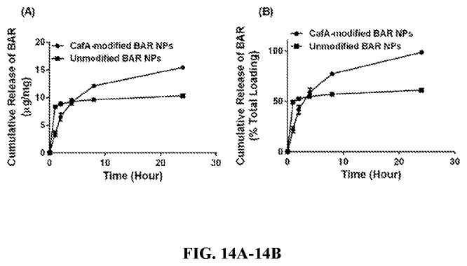

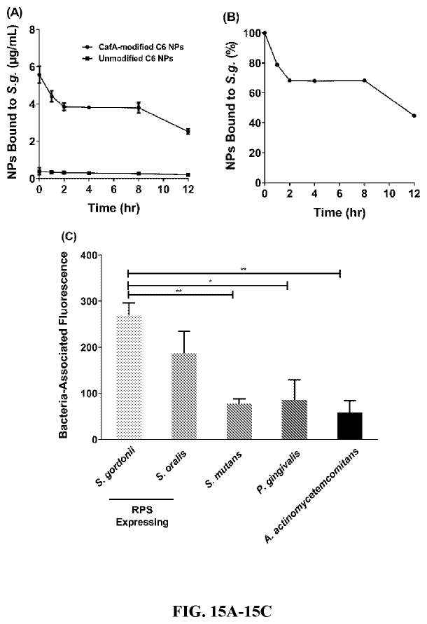

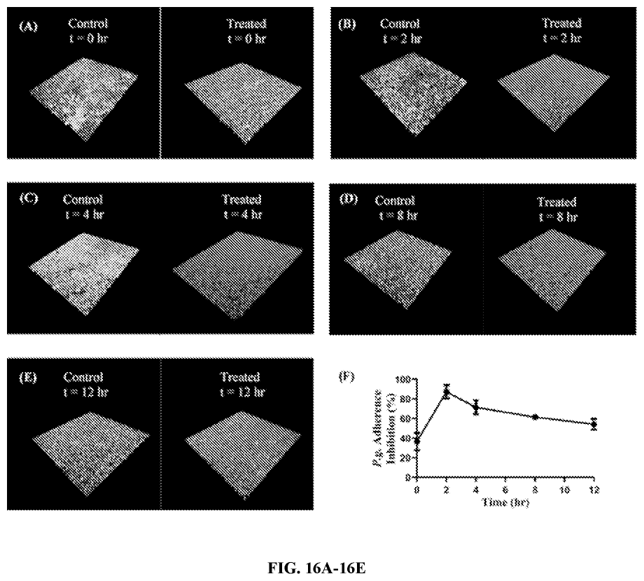

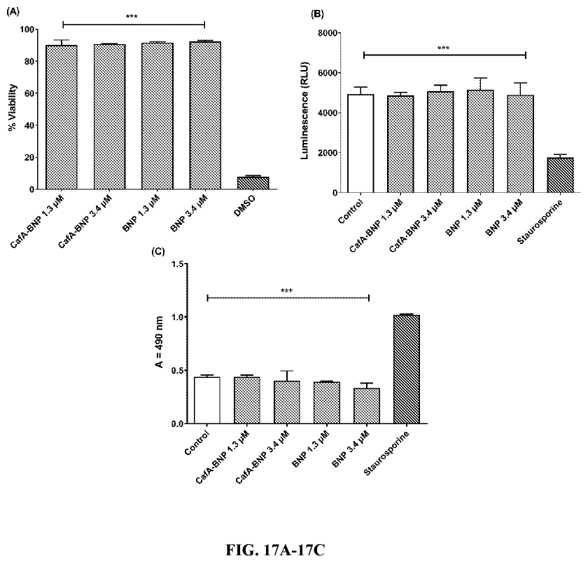

Oxidative DNA Damage: The number of AP sites was determined as an oxidative stress marker for cells treated with free BAR or 10:90 PLGA:PEO BAR-EFs (1.3 or 3.4 μM). As shown in FIG. 10, free BAR or BAR-EF treated (1.3 or 3.4 μM) cells demonstrated no change in the number of AP sites relative to medium-only treated cells, while cells treated with 2 mM H₂O₂ exhibited a significant increase in the number of AP sites relative to free BAR, BAR-EFs (1.3 or 3.4 μM), and medium-only treated cells (***, P≤0.001). These results suggested that neither free BAR nor BAR-EFs (1.3 or 3.4 μM) induce oxidative stress in TIGK cells.

### Part B—Assessment of Targeted BAR-Encapsulated NPs Against Oral Biofilms Example 17-CafA Expression and Purification

CafA synthesis was done by isolating the genomic DNA of Actinomyces oris (ATCC® 43146™) from 10 mL of an overnight culture using the Wizard Genomic DNA purification kit (Promega, Madison WI) as specified by manufacturer. The cafA gene was amplified by PCR using 200 ng of genomic DNA as the template and 30 pmol each of the following primers: Forward: 5′-AAG GAT CCC TGA GGC CGT TCA-3′ (SEQ ID NO:2); Reverse: 5′-CCG GAA TTC TAC GAC TTG CGG TTG GAG-3′ (SEQ ID NO:3). PCR amplification was conducted by denaturation at 94° C. for 2 min, annealing of primers and template at 63° C. for 30 s, strand extension at 72° C. for 2 min 45 s for 30 cycles, followed by a final extension cycle at 72° C. for 5 min.

The PCR product was subsequently electrophoresed in 1% agarose at 90 V for 40 min and the cafA band was excised and purified using the gel purification kit (Qiagen). The purified cafA DNA (1 μg) and a sample of the pGEX-6p-1 expression vector (0.5 μg) were digested with BamHI and EcoRI overnight at 37° C. Prior to ligation, 50 μL of the digested vector were dephosphorylated with 4 μL calf intestinal alkaline phosphatase (NEB) at 37° C. for 30 min. Subsequently, 3 μL of protease K were added and incubated for 30 min at 50° C. to terminate the reaction. The vector and cafA fragments were purified using the DNA clean and concentrator kit (Zymoresearch) and ligated with T4 ligase (New England Biolabs® Inc.). Ligation reactions were conducted according to the protocol provided by the manufacturer. Briefly, 5 μL cafA (80 ng), 3 μL pGEX-6p-1 (28 ng), 1 μL ligase buffer, and 1 μL T4 ligase were mixed and the ligation was carried out overnight at room temperature.

The ligation mixture was initially transformed into E. coli Top10. Fifty μL of competent E. coli Top10 were incubated with 5 μL of ligation mixture on ice for 30 min, then the sample was heat shocked at 42° C. for 45 s and placed on ice for 2 min. Two hundred μL of SOC media were added, the sample was incubated at 37° C. for 1 hr and plated on LB agar. After overnight incubation at 37° C., single colonies were selected and cultured in 5 mL LB broth supplemented with 100 μg ampicillin. Plasmid purification was carried out using the miniprep kit (Qiagen) and the cafA insert was excised and confirmed by sequencing.

For CafA expression, the purified cafA plasmid was transformed into E. coli BL21 using the transformation protocol described above. After selecting and confirming the appropriate transformant, 400 mL of LB broth was inoculated with 10 mL of an overnight culture and incubated to OD₆₀₀ of 0.5. Protein expression was induced by the addition of 0.5 mM IPTG and the culture was then incubated at 18° C. for 17 hr. After centrifugation at 4,250×g, the cell pellet was suspended in 40 mL 50 mM Tris, 100 mM NaCl, 1 mg/mL lysozome, 10 μg/mL DNase I, 400 μL protease inhibitor cocktail, 10 mM CHAPS, and incubated overnight at 4° C., followed by an additional 2 hr at 25° C. The cell suspension was then sonicated for 2 min on ice.

CafA purification was carried out with the Pierce GST Spin Purification Kit (Thermo Fisher). Seventeen mL of crude cell lysate were bound to the GST column for 2 hr at room temperature and the column was then centrifuged to remove unbound protein according to the specifications of the manufacturer. After washing the column with loading buffer, the GST tag was cleaved by the addition of 50 μL precision protease (GE Health) and overnight incubation at 4° C. Released CafA was then collected by centrifugation. The sample was then sequentially dialyzed against 30 mM, 20 mM, and 10 mM Tris for 2 hr each. CafA purity was determined by PAGE gels and protein concentration was determined using the BCA assay (Pierce).

### Example 18—Peptide Synthesis

BAR peptide consists of residues 1167 to 1193 of the SspB surface protein expressed by S. gordonii and is composed of the following amino acid sequence: NH₂-LEA APK KVQ DLL KKA NIT VKG AFQ LFS-COOH (SEQ ID NO: 1). To visualize and quantify the release of the peptide from CafA-modified NPs, 6-carboxyfluorescien was covalently attached to the F-amine of the lysine residue (underlined above) in the peptide sequence above to produce fluorescent BAR (F-BAR). Functional studies of CafA-modified NPs were carried out using NPs encapsulating unlabeled BAR peptide. Both labeled and unlabeled BAR peptide were synthesized by Biosynthesis, Inc. (Lewisville, TX) and were obtained with greater than 94% purity.

### Example 19—Growth of Bacterial Strains

Porphyromonas gingivalis ATCC 33277 was cultured in Trypticase soy broth (TSBY) media (Difco Laboratories Inc., Livonia, MI, USA) supplemented with 0.5% (w/v) yeast extract, 1 μg/mL menadione, and 5 μg/mL hemin. The growth medium was reduced for 24 hr in an anaerobic chamber (10% CO₂, 10% H₂ and 80% N₂). Twenty mL of reduced media was subsequently inoculated with 2 mL of an overnight P. gingivalis culture and incubated under anaerobic conditions for 48 hr at 37° C. Strains including S. gordonii DL-1, S. oralis SO34, S. mutans KPSP2 and A. actinomycetemcomitans 652 were cultured aerobically without shaking in brain-heart infusion (BHI) broth supplemented with 1% yeast extract for 16 hr at 37° C.

### Example 20—Conjugation of CafA Protein with Palmitic Acid

CafA-palmitate was synthesized as previously described (see, e.g., Fahmy et al., 2005, Biomaterials, 26:5727036; Steinbach, 2015, Cell Mol. Life Sci., 72:469-503; Steinback et al., 2012, J. Control Release, 162:102-10). Briefly, 2 mg of purified CafA was dissolved in 1.2 mL of 2% (w/v) sodium deoxycholate (NaDC) in phosphate-buffered saline (137 mM NaCl, 2.7 mM KCl, 1.8 mM KH₂PO₄, 10 mM Na₂HPO₄; PBS; pH=7.4) and warmed to 37° C. Next, a solution with 14-fold molar excess of the palmitic acid-N-hydroxysuccinimide ester (NHS-palmitic acid; Sigma-Aldrich, St Louis, MO, USA) was prepared by dissolving NHS-palmitic acid in 2% (w/v) NaDC at 0.125 mg/mL. The solution was sonicated until well mixed in an ultrasonic bath and 800 μL of this solution was added dropwise to the reaction vial containing CafA and reacted overnight at 37° C. To remove excess fatty acid and hydrolyzed ester, reactants were dialyzed against 1.2 L of PBS with 0.15% deoxycholate, using a 3,500 molecular weight cut-off dialysis tube. After overnight dialysis at 37° C., CafA-palmitate was stored at 4° C. until use.

### Example 21—Synthesis of CafA-Modified Blank NPs

Nanoparticles were synthesized using poly(lactic-co-glycolic acid) (PLGA) carboxyl-terminated polymer (0.55-0.75 dL/g inherent viscosity; LACTEL®). To formulate CafA-modified nanoparticles, a previously described oil-in-water (o/w) single emulsion technique was used (see, e.g., Sims et al., 2016, J. Nanobiotech., 15:33; Steinbach et al., 2016, Acta Biomater., 30:49-61). Briefly, 100 mg PLGA was dissolved in 2 mL dichloromethane (DCM) by overnight incubation at 25° C. The next day, 2 mL of 5% (w/v) polyvinyl alcohol (PVA) was added to 2 mL CafA-palmitate solution. This solution was vortexed and 2 mL of PLGA/DCM solution was added to it in a dropwise manner. The resulting solution was ultrasonicated and excess DCM was evaporated by adding the solution to 50 mL of 0.3% (w/v) PVA and mixing using a magnetic stir bar for 3 hr. After evaporation, the NP solution was centrifuged at 13,000 rpm (20,442×g) at 4° C. for 10 min. The supernatant, containing unreacted CafA conjugates was discarded, and the NPs were washed twice with deionized water (diH2O) followed by centrifugation at 13,000 rpm (20,442×g) at 4° C. for 10 min. After washing, CafA-modified NPs were suspended in 5 mL of diH2O, freeze dried at −80° C., and lyophilized under 10 mTorr for 48 hr.

### Example 22—Synthesis of CafA-Modified NPs Encapsulating C6/F-BAR/BAR

For our experiments, three different types of CafA-modified NPs were synthesized. CafA-modified NPs encapsulating the fluorescent dye, Coumarin 6 (C6), were synthesized to assess the binding functionality of CafA surface modification. CafA-modified NPs encapsulating fluorescent BAR (F-BAR) were synthesized to determine the loading and controlled release characteristics of BAR from surface-modified NPs, and CafA-modified NPs encapsulating unlabeled BAR were synthesized to determine the efficacy of NP-mediated inhibition of P. gingivalis adherence to streptococci using a two-species biofilm model.

CafA-modified NPs encapsulating C6 or BAR were synthesized similar to above using a previously described w/o/w double emulsion solvent evaporation technique (see, e.g., Steinbach, 2015, Cell. Mol. Life Sci., 72:469-503; Steinbach et al., 2012, J. Control Release, 162:102-10). Briefly, C6 was dissolved overnight in 200 μL DCM at a concentration of 15 μg/mg PLGA. In parallel, 100 mg of PLGA crystals were dissolved in 2 mL of DCM by overnight incubation at 25° C. The following day, the C6 DCM solution was first emulsified in the PLGA/DCM solution by vortexing, followed by ultrasonication to achieve a homogeneous suspension. Next, the homogeneous suspension was added dropwise to a mixture of 2 mL of 5% (w/v) polyvinyl alcohol (PVA) and 2 mL CafA-palmitate solution while vortexing, followed by ultrasonication. Excess DCM was evaporated and NPs were collected as described above. CafA-modified NPs encapsulating F-BAR/BAR were synthesized using a similar approach. All synthesis reactions were protected from exposure to light. For the synthesis of CafA-modified NPs encapsulating either F-BAR or unlabeled BAR, the peptide was dissolved in 200 μL Tris EDTA buffer (VWR; 100 mM Tris HCl, 10 mM EDTA at a pH of 8.0; TE buffer) at a concentration of 43 μg BAR/mg PLGA.

### Example 23—NP Characterization: NP Morphology, Size and Zeta Potential

Unhydrated NP morphology, diameter, and size distribution were determined using scanning electron microscopy (SEM, XL-30 ESEM-FEG SEM, FEI Company, USA). Lyophilized NPs were mounted on carbon tape and sputter coated with a thin layer of gold/palladium. Average diameters of 500 particles were determined from SEM images (n=3, 50 NPs measured per field of view) using image analysis software (ImageJ, National Institutes of Health, version 1.5a, ImageJ.nih.gov). The zeta potential was measured to determine surface charge of hydrated NPs. Briefly, a 1 mg/mL samples of unmodified PLGA NPs and CafA-modified NPs in diH2O were prepared. After vortexing and sonication, samples were diluted at a 1:10 ratio in diH2O. One mL was aliquoted to the cuvette for analysis using Brookhaven Instrument Corporation 90 plus.

### Example 24—Surface Density of CafA

CafA-modified NPs were synthesized using varying input concentrations of CafA protein (5 to 80 μg/mg polymer). For each input condition, the resulting concentration of CafA conjugated to the NP surface was measured using the microBCA assay (Pierce). CafA-modified NPs (1 mg) were suspended in 1% dimethyl sulfoxide (DMSO) in PBS. Aliquots (100 μL) of the NP samples were analyzed in triplicate in a microtiter plate and NP-associated absorbance was measured by spectrophotometry at a wavelength of 562 nm. The concentration of CafA was determined by comparing absorbance values to a known standard curve of CafA and subtracting the background absorbance values of unmodified NPs (control group).

### Example 25—Loading and Release Kinetics of BAR Peptide from Unmodified and CafA-Modified NPs

Nanoparticles modified with an intermediate density of CafA (20 μg/mg polymer) were selected for subsequent characterization and functionality studies. To determine BAR peptide loading, approximately 2 mg CafA-modified NPs encapsulating F-BAR were dissolved in 1 mL DMSO. Aliquots (100 μL) of the NP samples were analyzed in triplicate in a microtiter plate and the amount of F-BAR in the dissolved solution was determined by measuring fluorescence (488/518 nm excitation/emission) and quantified by comparing these values to a known standard curve of F-BAR.

To analyze the release kinetics of F-BAR, aliquots of ˜1 to 3 mg CafA-modified and unmodified NPs encapsulating F-BAR were incubated in microcentrifuge tubes containing 1 mL PBS (pH 7.4) at 37° C. with gentle horizontal agitation. At fixed time points (1, 2, 4, 8, and 24 hr) after the initial suspension, the samples were centrifuged at 18,900×g and the supernatant was collected. The pelleted NPs were then suspended in fresh PBS and incubated until the next time point. The amount of F-BAR in the supernatant was determined by measuring fluorescence (488/518 nm excitation/emission) and quantified by comparing these values to a known standard curve of F-BAR.

### Example 26—Determination of Surface Modification Functionality

Duration of CafA-Modified NP Adhesion

The functionality of surface modification and preservation of the function of CafA protein during NP synthesis was analyzed using two approaches. To determine the duration of retention of CafA-modified C6 NPs on S. gordonii cells, S. gordonii was cultured as previously described and bacterial cells were harvested by centrifuging 10 mL of culture at 3,700×g for 5 min. The supernatant was discarded and the pelleted cells were suspended in 1 mL of PBS. The OD at 600 nm of the cell suspension was adjusted to 0.2 and 100 μL of the S. gordonii cell suspension was added to each well of a 96-well microtiter plate and incubated overnight at 4° C. After removing unbound cells, the wells were blocked for non-specific binding with 300 μL of 0.3% bovine serum albumin (BSA) for 1 hr. Thereafter, the microtiter plate was washed three times with 1×PBS containing 0.05% Tween (PBST). Immobilized S. gordonii cells were then incubated with 100 μL of CafA-modified C6 NPs (0.25 mg/mL), unmodified C6 NPs (0.25 mg/mL, control group) or PBST in the absence of NPs in triplicate for 1 hr on a rocker platform. After washing three times with PBST, the cell-associated fluorescence was measured using Synergy HT reader (BioTek, Winooski, VT, USA) (485/520 nm excitation/emission). After subtracting the control fluorescence (S. gordonii incubated with PBST), the initial reading (at t=0) was defined as 100% binding. After obtaining the initial reading, 100 μL of PBST was added to each well and at fixed time points (1, 2, 4, 8, 12 hr), the PBST was removed and the cell-associated fluorescence that remained was measured. Subsequently, an additional 100 μL aliquot of fresh PBST was added per well and incubated until the next time point was reached.

Specificity of CafA-Modified NP Adhesion

To determine the specificity of CafA adhesion, the adherence of CafA-modified C6 NPs to S. gordonii DL-1, S. oralis SO34, S. mutans KPSP2, P. gingivalis ATCC 33277 and A. actinomycetemcomitans 652 cells was measured. CafA binds to the GalNAcβ1-3Gal motif of the receptor polysaccharides (RPS) found only on commensal oral streptococci such as S. gordonii and S. oralis. It does not bind to bacteria lacking this motif and therefore, S. mutans, P. gingivalis, and A. actinomycetemcomitans were selected as negative controls. Each of the organisms was cultured and harvested as described previously, and the final OD at 600 nm for each cell suspension was adjusted to 0.2. The bacterial cells were immobilized on a 96-well microtiter plate as described above and after overnight incubation, wells were blocked for non-specific binding with 300 μL of 3% bovine serum albumin (BSA) for 1 hr. The plate was washed three times with PBST, and immobilized bacterial cells were incubated with 100 μL of CafA-modified C6 NPs (0.25 mg/mL), unmodified C6 NPs (0.25 mg/mL) or with PBST in triplicate for 1 hr on a rocker platform. The microtiter plate was again washed three times with PBST, and cell-associated fluorescence was measured (485/520 nm excitation/emission). To determine the final cell-associated fluorescence, the readings obtained from bacteria incubated with unmodified C6 NPs (control) were subtracted from that of bacteria incubated with CafA-modified C6 NPs. Data were analyzed using an independent t-test.

### Example 27—CafA-Modified NP-Mediated Inhibition of P. gingivalis Adherence to Streptococci

S. gordonii DL-1 was cultured as previously described and bacterial cells were harvested by centrifuging 10 mL of culture at 3,700×g for 5 min. The supernatant was discarded, and the pelleted cells were suspended in 1 mL of 1×PBS in a microcentrifuge tube. The cells were labeled with 20 μL of 10 mM hexidium iodide (Thermo Fisher Scientific) for 15 min on a rocker platform at room temperature. The microcentrifuge tube was covered with foil, centrifuged at 3,700×g for 5 min, and the pelleted cells were suspended in 1 mL of 1×PBS. The OD at 600 nm was measured as previously described and adjusted to 0.8. One mL of the resulting cell suspension was added to each well of a 12-well microtiter plate containing a glass coverslip. The cells were incubated overnight under anaerobic conditions on a rocker platform and protected from light.

On the following day, the wells were washed to remove unbound S. gordonii cells. The immobilized S. gordonii cells were incubated with CafA-modified BAR NPs (treatment) or CafA-modified blank NPs (control) at a concentration of 240 μg/mL for different durations (0, 2, 4, 8, 12 hr) on a rocker platform. Due to the 50% inhibitory concentration (IC₅₀) of free BAR peptide equivalent to 1.3 μM or ˜4 μg, and NP loading results, we calculated that 240 μg CafA-modified BAR NPs would encapsulate an equivalent amount of BAR. At the initial time point (t=0), CafA-modified NPs and P. gingivalis were added simultaneously in triplicate. At each subsequent time point (t=2, 4, 8, 12 hr) the supernatant containing the unbound NPs and released BAR was removed and P. gingivalis was added in triplicate to the control and treatment plates as described below.

P. gingivalis ATCC 33277 was cultured and harvested as previously described. P. gingivalis was labeled with 15 μL of 5-(6) carboxyfluorescien-succinylester (4 mg/mL) for 30 min, centrifuged at 3,700×g for 2 min and the pelleted cells were suspended in 1 mL of 1×PBS. The OD at 600 nm was measured and adjusted to 0.4. At each time point, 1 mL of labeled P. gingivalis cell suspension (OD 0.4) was added to the treatment and control plates in triplicate.

The plates were incubated at 37° C. for 24 hr under anaerobic conditions. The subsequent day the supernatant was removed, the wells were washed with 1×PBS to remove unbound bacterial cells, and adherent cells were fixed with 4% (w/v) paraformaldehyde. The coverslips were mounted on a glass slide using clear nail polish. The prepared slides were stored at 4° C.

The dual-species biofilms were visualized using a LEICA SP8 confocal microscope (Leica Microsystems Inc., Buffalo Grove, IL) under 60× magnification. Three-dimensional z-stack biofilm images were obtained using a z-step size of 0.7 μm. Images were analyzed using Volocity software (version 6.3; Perkin Elmer, Waltham, MA, USA) to quantify the bacterial populations by quantifying fluorescence (S. gordonii—red, P. gingivalis—green). Adherence of P. gingivalis to streptococci was determined by measuring the green to red fluorescence ratio (GR). Inhibition at each time point was analyzed in triplicate and three independent frames were obtained for each well. The percentage of P. gingivalis inhibition was calculated using the formula: (1−GR treatment/GR control)*100. The mean and standard deviation of inhibition at each time point was calculated.

### Example 28—Determination of CafA-Modified and Unmodified BAR-NP In Vitro Cytotoxicity

MTT Assay

TIGK cells were seeded in 12-well plates at a density of 6×10⁴ cells in 1 mL media per well, and incubated for 24 hr to allow for 60 to 70% confluency and sufficient adhesion. Cells were treated with 1.3 or 3.4 μM of CafA-modified BAR-NPs or unmodified BAR-NPs. After 24 hr, 100 μL of MTT solution (3-(4,5-dimethylthiazol-2-yl)-2,5-diphenyltetrazolium bromide, 10% of total volume) was added to the media of all samples. The solution was incubated at 37° C. for 4 hr. After this period, 550 μL of lysis buffer (50% of total volume) was added to the media of each well and plates were incubated overnight. The absorbance of each well was read at 570 nm, and the sample absorbance was normalized to the absorbance of untreated cells (media only). Treatment with 10% DMSO media (100 μL DMSO in 900 μL media) was used as a positive control for cell death.

ATP Assay

Total ATP levels in cell culture were assessed by using the CellTiter-Glo reagent (Promega, Madison WI), as described by the manufacturer. TIGK cells were seeded at a density of 6×10⁴ cells in 1 mL media per well and incubated at 37° C., 5% CO₂ for 24 hr in a 12-well flat bottom plate. Cells were then incubated with CafA-modified BAR-NPs or unmodified BAR-NPs (1.3 or 3.4 μM) for 24 hr at 37° C. in 5% CO₂. Cells were then lysed with 500 μL of 0.1% Triton X-100 for 30 min at 37° C. The lysates were collected and centrifuged at 1,000×g for 10 min at 4° C., and 50 μL of supernatant was mixed with 50 μL of CellTiter-Glo reagent. Samples were incubated at ambient temperature for 10 min in a black 96-well plate in the dark. Total luminescence was measured with a Victor 3 luminometer (Perkin-Elmer, Inc). Cells incubated with 1 ng of staurosporine or with medium-only served as positive and negative controls for cell death, respectively.

LDH Assay

Cell membrane leakage was measured by the release of lactate dehydrogenase (LDH). Extracellular LDH was quantified using a CytoTox96® non-radioactive cytotoxicity assay (Promega, Madison WI) as described by the manufacturer. TIGK cells were plated at density of 6×10⁴ cells in 1 mL media per well in a 12-well flat bottom plate, and incubated at 37° C., 5% CO₂ for 24 hr. CafA-modified BAR NPs or unmodified BAR NPs (1.3 or 3.4 μM) were added to cells in triplicate for 24 hr at 37° C. in 5% CO₂. Fifty microliters of supernatant from CafA-modified BAR NP- and unmodified BAR NP-treated (1.3 and 3.4 μM) cells were added to the LDH substrate and incubated at room temperature for 30 min. The reactions were subsequently terminated by adding 50 μL of stop solution. LDH activity was determined by measuring the optical density of the solution at 490 nm. Cells treated with staurosporine or with medium-only served as positive and negative controls for cell death, respectively.

Data from each of the toxicity tests were analyzed using ANOVA after passing Bartlett's and Brown-Forsythe tests for homogeneity of variances using GraphPad InStat (La Jolla, CA). A pair-wise, parametric analysis of variance using a Bonferroni multiple comparison post-test was used to determine the statistical significance between the individual groups. A P-value of ≤0.05 was considered to be statistically significant.

### Example 29—NP Characterization: NP Morphology, Size and Zeta Potential

The morphology of CafA-modified BAR NPs, relative to unmodified BAR NPs, is shown in FIG. 12. CafA-modified BAR NPs demonstrated a spherical morphology and smaller size relative to unmodified BAR NPs. The average unhydrated diameters of CafA-modified BAR NPs and unmodified BAR NPs measured from SEM images were 89.7±26.3 nm and 165.8±33.4 nm, respectively, while the corresponding size ranges were 68.7 to 143.5 nm and 86.7 to 209.6 nm. The zeta potentials of CafA-modified NPs and unmodified NPs were −38.69±0.21 and −39.58±0.26, respectively. The similarity in zeta potential of CafA-modified and unmodified NPs is attributed to the neutral charge of CafA (isoelectric potential=7.1).

### Example 30—Quantification of CafA Surface Density

The concentration of CafA conjugated to the NP surface was measured using the microBCA assay. The total protein content ranged from 3 to 36 μg CafA/mg polymer and varied directly with the input concentration of CafA (5 to 80 μg CafA/mg polymer) used during synthesis. The conjugation efficiency ranged from 45 to 79%, with higher conjugation efficiency observed at lower concentrations (FIG. 13, Table 2). Although NP surface saturation was not achieved with these input concentrations, the results suggest that an increased surface density may be attained with higher CafA input.

TABLE 2
Surface density of CafA input versus
CafA conjugation concentration.

| Input Concentration | Output Concentration | Conjugation Efficiency |
|---|---|---|
| (μg CafA/mg NP) | (μg CafA/mg NP) | (%) |
| 5 | 2.9 ± 0.1 | 58 |
| 10 | 7.9 ± 1.9 | 79 |
| 20 | 14.4 ± 2.6 | 72 |
| 40 | 25.4 ± 2.2 | 64 |
| 80 | 36.3 ± 3.5 | 45 |

### Example 31—Loading and Release Kinetics of BAR Peptide from Unmodified and CafA-Modified NPs

Nanoparticles modified with an intermediate density of CafA (20 ug/mg polymer) were selected for subsequent characterization and functionality studies, as they represent a practical minimum modification density that in preliminary studies (and results here) provided a therapeutically-relevant concentration of BAR release. To determine the loading of BAR peptide in CafA-modified and unmodified NPs, the amount of F-BAR from solubilized NPs was quantified by measuring fluorescence (488/518 nm excitation/emission) and comparing these values to an F-BAR standard curve. Loading experiments showed that CafA-modified and unmodified NPs encapsulated 15.73±1.9 μg and 16.95±0.8 of BAR per mg of NP respectively, corresponding to loading efficiencies of 37% and 39%, suggesting that CafA surface modification at this density had minimal effect on BAR loading.

In release experiments, sample eluates were taken 1, 2, 4, 8, and 24 hr after incubation in PBS. The overall release trends showed that CafA-modified NPs demonstrated slower release of BAR, relative to unmodified NPs; however, inhibitory concentrations of BAR peptide (2 to 4 μg/mg NP) were released from CafA-modified NPs at each of the measured time points up to 8 hr. For unmodified NPs, more rapid release profiles were observed, with greater than 50% of BAR peptide released within 1 hr and a plateau in release after. After 2, 4, 8 and 24 hr, less than 1 μg of peptide/mg NP was released from the unmodified NPs (FIG. 14), demonstrating inadequate, non-inhibitory levels of release. In comparison, CafA-modified NPs released 23% of BAR during the first hour, and inhibitory concentrations (3.1, 2.7, 2.9, and 3.3 μg/mg) of BAR peptide after 2, 4, 8, and 24 hr. Cumulatively, after 24 hr, BAR peptide (15.5 μg/mg) was completely released from CafA-modified BAR NPs, whereas only 61% of the encapsulated BAR (10.3 μg/mg) was released from unmodified BAR-encapsulated NPs. Thus, after 24 hr, the total quantity of BAR released from CafA-modified NPs was significantly, and surprisingly, higher than the amount of BAR released from unmodified NPs.

### Example 32—Determination of Surface Modification Functionality

Duration of CafA-Modified NP Adhesion

CafA-modified C6 NPs were administered to immobilized S. gordonii cells for 1 hr, after which unbound NPs were washed and cell-associated fluorescence was measured, to determine the amount of NPs that initially bound to S. gordonii (t=0). As shown in FIG. 15A, at the initial time point (t=0), CafA-modified NPs bound to S. gordonii at a 14.4-fold higher concentration (5.6 μg/mL), relative to unmodified NPs (0.4 μg/mL). After 1 hr of initial binding (t=1), 79% of CafA-modified NPs remain bound to S. gordonii and after 2 hr (t=2), 69% of CafA-modified NPs remain bound to S. gordonii, maintaining binding through 8 hr. Between 8 and 12 hr, an additional 23% NP dissociation was observed, resulting in 45% (2.5 μg/mL) of CafA-modified and 52% of unmodified NPs (0.2 μg/mL) associated with S. gordonii. After 12 hr, this resulted in a 12.4-fold higher concentration of CafA-modified NPs bound to S. gordonii (FIG. 15B). These results indicate that the ratio of CafA-modified to unmodified NPs bound to S. gordonii was maintained after the first wash and suggest that the targeting moiety, CafA, improves the binding efficiency of unmodified NPs and enhances their retention by significantly increasing the concentration of CafA-modified NPs that bind to S. gordonii relative to unmodified NPs.

Specificity of CafA-Modified NP Adhesion

While the overall adhesion and retention of CafA-modified NPs to S. gordonii is important, we sought to assess the differences in CafA-modified NP binding across several bacteria to determine the specificity of NP adhesion to RPS-expressing bacteria (i.e., S. gordonii and S. oralis). The adherence of CafA-modified NPs to S. gordonii DL-1 was measured relative to S. oralis SO34, S. mutans KPSP2, P. gingivalis ATCC 33277 and A. actinomycetemcomitans 652 cells (FIG. 15C). For CafA-modified NPs, no statistical significance was observed in the bacteria-associated fluorescence between commensal oral streptococci, S. gordonii DL-1 and S. oralis SO34, that express the GalNAcβ1-3Gal motif. However, compared to non-commensal bacterial groups that lacked the CafA binding motif (S. mutans, P. gingivalis, and A. actinomycetemcomitans), S. gordonii DL-1 associated fluorescence was 3.5-fold higher relative to S. mutans KPSP2, 3.2-fold higher relative to P. gingivalis ATCC 33277, and 4.6-fold higher relative to A. actinomycetemcomitans 652. The fluorescence associated with S. gordonii DL-1 was found to be statistically significant (P≤0.05) relative to S. mutans KPSP2, P. gingivalis ATCC 33277, and A. actinomycetemcomitans 652. No statistical significance in bacteria-associated fluorescence was observed between non-commensal bacterial groups. Together these results indicate that CafA-modified NPs bind to RPS-expressing commensal streptococci at a higher concentration relative to non-commensal organisms, demonstrating specificity of adherence.

### Example 33—CafA-Modified NP-Mediated Inhibition of P. gingivalis Adherence to Streptococci

Functional inhibition assays were performed to determine the impact of CafA-modified BAR NPs on the inhibition of P. gingivalis adhesion to S. gordonii. Immobilized S. gordonii were incubated with CafA-modified BAR NPs (treatment group) or CafA-modified blank NPs (control) for different durations (0, 2, 4, 8, 12 hr). At the initial time point (t=0), CafA-modified NPs and P. gingivalis were added simultaneously in triplicate. At each subsequent time point (t=2, 4, 8, 12 hr) the supernatant containing the unbound NPs and released BAR was removed and P. gingivalis was added in triplicate to the control and treatment plates. The plates were incubated for 24 hr and the formed biofilms were visualized using confocal microscopy. Representative images of treatment and control biofilms are shown in FIG. 16A-16E. At each time point (t=0, 2, 4, 8 and 12 hr), P. gingivalis adherence to S. gordonii was significantly reduced in the presence of CafA-modified BAR NPs, relative to control CafA-modified blank NPs (FIG. 16F, Table 3). After initial administration (t=0), P. gingivalis adherence was inhibited by 36.6±8.8%. After 2 hr, more than 70% inhibition was observed and maintained for up to 4 hr relative to control CafA-modified blank NPs. After 8 and 12 hr, time frames relevant to oral administration regimens, P. gingivalis binding to S. gordonii was inhibited by 61.5±2.7% and 54.3±5.6%, demonstrating the potential of CafA-modified BAR NPs to significantly inhibit P. gingivalis adherence to S. gordonii. Hence, CafA-modified NPs maintained over 50% inhibition for 12 hr relative to control NPs.

TABLE 3
Percent inhibition of P. gingivalis adherence
to S. gordonii at various time points

| Time Point (hr) | Adherence Inhibition of P.g. (%) |
|---|---|
| t = 0 | 36.6 ± 8.8 |
| t = 2 | 87.4 ± 6.9 |
| t = 4 | 71.4 ± 7.2 |
| t = 8 | 61.5 ± 2.7 |
| t = 12 | 54.3 ± 5.6 |

### Example 34—Determination of CafA-Modified and Unmodified BAR-NP In Vitro Cytotoxicity

MTT Assay

To assess the effect of CafA-modified BAR NPs or unmodified BAR NPs on the viability of TIGK cells, cells were incubated with CafA-modified or unmodified BAR NPs (1.3 or 3.4 μM) for 2 d and viability was measured using the MTT assay. As shown in FIG. 17A, treated cells exhibited little loss in viability, suggesting CafA-modified and unmodified BAR NPs are biocompatible with TIGK cells when applied for up to 2 d.

ATP Assay

Cytotoxicity was also determined by assessing the metabolic activity of TIGK cells by measuring ATP levels. As shown in FIG. 17B, staurosporine-treated cells demonstrated significantly lower levels of ATP (P≤0.001) than were observed for untreated; CafA-modified BAR NP-treated; and unmodified BAR NP-treated cells.

LDH Assay

Since some peptides are known to damage the cell membrane, we next measured LDH activity as a marker for cell membrane integrity after treatment with CafA-modified or unmodified BAR NPs. FIG. 17C shows that LDH levels released from cells treated with CafA-modified or unmodified BAR NPs (1.3 or 3.4 μM) were negligible when compared to control (medium-treated) cells. In contrast, LDH activity released from cells treated with staurosporine was significantly higher than control or treated cells (P≤0.001), indicating that CafA-modified BAR NPs or unmodified BAR NPs do not compromise cell membrane integrity.

It is to be understood that, while the methods and compositions of matter have been described herein in conjunction with a number of different aspects, the foregoing description of the various aspects is intended to illustrate and not limit the scope of the methods and compositions of matter. Other aspects, advantages, and modifications are within the scope of the following claims.

Disclosed are methods and compositions that can be used for, can be used in conjunction with, can be used in preparation for, or are products of the disclosed methods and compositions. These and other materials are disclosed herein, and it is understood that combinations, subsets, interactions, groups, etc. of these methods and compositions are disclosed. That is, while specific reference to each various individual and collective combinations and permutations of these compositions and methods may not be explicitly disclosed, each is specifically contemplated and described herein. For example, if a particular composition of matter or a particular method is disclosed and discussed and a number of compositions or methods are discussed, each and every combination and permutation of the compositions and the methods are specifically contemplated unless specifically indicated to the contrary. Likewise, any subset or combination of these is also specifically contemplated and disclosed.

1. A composition comprising a BAR polypeptide encapsulated within a nanoparticle.

2. The composition of claim 1, wherein the nanoparticle is comprised of at least one hydrophobic polymer and/or at least one hydrophilic polymer.

3. The composition of claim 1, wherein the at least one hydrophobic polymer is selected from poly(lactic-co-glycolic) acid (PLGA), poly(L-lactic acid) (PLLA), polycaprolactone (PCL), polyurethane, silicone, and combinations thereof.

4. The composition of claim 1, wherein the at least one hydrophilic polymer is selected from polyethylene oxide (PEO), polyvinyl pyrrolidone, polyvinyl alcohol, and combinations thereof.

5. The composition of claim 1, wherein the nanoparticles have an average diameter of about 50 nm to about 100 nm.

6-16. (canceled)

17. The composition of claim 1, wherein the BAR polypeptide has the sequence shown in SEQ ID NO:1.

18. A method of preventing or reducing an interaction between Porphyromonas gingivalis and streptococci spp. in the oral cavity of an individual, comprising:
administering the composition of claim 1 to the oral cavity of the individual,
thereby preventing or reducing the interaction between Porphyromonas gingivalis and streptococci spp. in the oral cavity of the individual.

19. The method of claim 18, wherein the composition releases inhibitory concentrations of BAR polypeptide for at least 2 hours.

20. The method of claim 18, wherein the composition is administered once per day.

21-24. (canceled)

25. A composition, comprising:
electrospun fibers comprised of at least one hydrophobic polymer and/or at least one hydrophilic polymer; and
a SspB Adherence Region (BAR) polypeptide from a streptococcal antigen I/II protein.

26. The composition of claim 25, wherein the at least one hydrophobic polymer is selected from poly(lactic-co-glycolic acid) (PLGA), poly(L-lactic acid) (PLLA), polycaprolactone (PCL), polyurethane, silicone, and combinations thereof.

27. The composition of claim 25, wherein the at least one hydrophilic polymer is selected from polyethylene oxide (PEO), polyvinyl pyrrolidone, polyvinyl alcohol, and combinations thereof.

28. The composition of claim 25, wherein the hydrophilic polymer is PLGA.

29. The composition of claim 25, wherein the ratio of hydrophobic to hydrophilic polymer is selected from at least 49:51, at least 20:80, or at least 10:90.

30-31. (canceled)

32. The composition of claim 25, wherein the at least one hydrophobic polymer or the at least one hydrophilic polymer is present in an amount of about 10%-18% w/w.

33. The composition of claim 25, wherein the diameter of the fibers is about 0.5 μm to about 1.5 μm.

34. (canceled)

35. The composition of claim 25, wherein the BAR polypeptide comprises residues 1167-1193 of the SspB protein sequence of S. gordonii.

36. The composition of claim 25, wherein the BAR polypeptide has the sequence shown in SEQ ID NO:1.

37. The composition of claim 25, wherein the BAR polypeptide is present at an amount of 1% w/w relative to the at least one hydrophobic polymer and/or the at least one hydrophilic polymer.

38-41. (canceled)

42. A method of preventing or reducing an interaction between Porphyromonas gingivalis and streptococci spp. in the oral cavity of an individual, comprising:
administering the composition of claim 25 to the oral cavity of the individual,
thereby preventing or reducing the interaction between Porphyromonas gingivalis and streptococci spp. in the oral cavity of the individual.

43. The method of claim 42, wherein the composition is administered once per day.

44. (canceled)

45. A method of making the composition of claim 1, comprising:
combining a solution comprising at least one hydrophobic polymer and/or at least one hydrophilic polymer with a BAR polypeptide with to produce a polymer-BAR polypeptide solution;
electrospinning the polymer-BAR solution to produce BAR polypeptide-incorporated fibers.

46. The method of claim 45, wherein the amount of BAR polypeptide in the solution is about 1.0 μM to about 5.0 μM.

47. The method of claim 45, wherein the amount of BAR polypeptide in the solution is about 1.3 μM to about 3.4 μM.

48. The method of claim 45, wherein the amount of BAR polypeptide in the solution is about 1% by weight.


SEQUENCE LISTING (6 sequences)

SEQ ID NO: 1 - protein, 27 aa, Streptococcus gordonii
LEAAPKKVQDLLKKANITVKGAFQLFS

SEQ ID NO: 2 - DNA, 21 bp, Artificial Sequence
  Synthetic oligonucleotide
aaggatccctgaggccgttca

SEQ ID NO: 3 - DNA, 27 bp, Artificial Sequence
  Synthetic oligonucleotide
ccggaattctacgacttgcggttggag

SEQ ID NO: 4 - protein, 1579 aa, Streptococcus gordonii
MNKRKEVFGFRKSKVAKTLCGAVLGAALIAIADQQVLADEVTETNSTANVAVTTTGNPATNLPEAQGEATEAASQSQAQAGSKDGALPVEVSADDLNKAVTDAKAAGVNVVQDQTSDKGTATTAAENAQKQAEIKSDYAKQAEEIKKTTEAYKKEVEAHQAETDKINAENKAAEDKYQEDLKAHQAEVEKINNANATAKAEYEAKLAQYQKDLAAVQKANEDSQLDYQNKLSAYQAELARVQKANAEAKEAYEKAVKENTAKNAALQAENEAIKQRNETAKANYDAAMKQYEADLAAIKKAKEDNDADYQAKLAAYQAELARVQKANADAKAAYEKAVEENTAKNTAIQAENEAIKQRNAAAKATYEAALKQYEADLAAVKKANEDSEADYQAKLAEYQTELARVQKANADAKAAYEKAVEDNKAKNAALQAENEEIKQRNAAAKTDYEAKLAKYEADLAKYKKELAEYPTKLKAYEDEQAKIKAALVELEKNKDQDGYLSKPSAQSLVYDSEPNAQLSLVTEGSFLKATAVDNAFKQDAAQYGKKNLQLDNLNVTYLEGAAATSTSMELYGNINDKSDWTTNVGNKTEVKWGSVLLERGQSVTATYTNLQNSYYNGKKISKIVYKYTVDPSSQFKNPSGKVWLGIFTDPTLGVFASAYTGQVEKDTSLFIKNEFTFYDENDQPINFDNALLSVASLNREHNSIEMAKDYSGTFIKISGSSIGEKNGMIYATKTLNFRKDQGGSRWTMYPRANEPGSGWDSSDAPNSWYGAGAISMSGPTNHVTVGATSATNVMSVAEMPQVPGRDNTEGKRPNIWYSLNGKIRAVNVPKITKEKPTPPVAPTEPQAPTYEVEKPLEPAPVAPTYENEPTPPVKTPDQPEPSKPEEPTYETEKPLEPAPVVPTYENEPTPPVKTPDQPEPSKPEEPTYETEKPLEPAPVAPTYENEPTPPVKTPDQPEPSKPEEPTYDPLPTPPLAPTPKQLPTPPVVPTVHFHYSNLLAQPQINKEIKNEDGVDIDRTLVAKQSIVKFELKTEALTAGRPKTTSFVLVDPLPTGYKFDLDATKAASTGFDTTYDEASHTVTFKATDETLATYNADLTKPVETLHPTVVGRVLNDGATYTNNFTLTVNDAYGIKSNVVRVTTPGKPNDPDNPNNNYIKPTKVNKNKEGLNIDGKEVLAGSTNYYELTWDLDQYKGDKSSKEAIQNGFYYVDDYPEEALDVRPDLVKVADEKGNQVSGVSVQQYDSLEAAPKKVQDLLKKANITVKGAFQLFSADNPEEFYKQYVATGTSLVITDPMTVKSEFGKTGGKYENKAYQIDFGNGYATEVVVNNVPKITPKKDVTVSLDPTSENLDGQTVQLYQTFNYRLIGGLIPQNHSEELEDYSFVDDYDQAGDQYTGNYKTFSSLNLTMKDGSVIKAGTDLTSQTTAETDATNGIVTVRFKEDFLQKISLDSPFQAETYLQMRRIAIGTFENTYVNTVNKVAYASNTVRTTTPIPRTPDKPTPIPTPKPKDPDKPETPKEPKVPSPKVEDPSAPIPVSVGKELTTLPKTGTNDATYMPYLGLAALVGFLGLGLAKRKED

SEQ ID NO: 5 - DNA, 4740 bp, Streptococcus gordonii
ttaatcttctttgcgttttgccagacctaatcctaggaaaccaacaagggcagccaagcctaagtaaggcatataagtcgcatcatttgttcctgtttttggaagtgttgttaattctttaccaacagaaacaggaattggtgctgaaggatcttcaacttttggactaggaacttttggttcttttggtgtctcaggtttgtcaggatcctttggttttggcgttggaattggtgtcggtttgtctggtgttcttggtattggagttgttgtacgtactgtgttagatgcataagcaaccttattaacagtatttacataagtattttcaaatgttccaatagcaattctgcgcatttgaaggtaggtttcagcttggaatggcgaatccaaactaatcttttgtaaaaagtcttccttgaaacgaacagttacaataccatttgtagcatctgtttcagcagtagtttgagatgttagatctgtacctgccttaatcactgaaccatctttcattgtcaagttcagagagctgaatgtcttgtaattaccagtatactgatcaccagcttggtcataatcatccacaaagctgtaatcttctaattcctcagaatgattttgtggaatgaggccaccaatcagacgatagttaaatgtttgatacaattgaactgtttgaccatccagattttcactagttggatctaggctcactgttacgtcttttttaggggtgattttcggtacgttgttaaccactacttctgtcgcataaccatttccgaaatcaatttgataggccttattttcatacttaccacctgtcttaccaaattcagacttaacagtcatcggatctgtaatgactagtgatgttccagttgctacatattgcttgtaaaattcttctggattatcagcagagaagagttggaaagcacccttaacagtaatgttagctttcttcaacaagtcttgaactttcttaggagcggcttctagactgtcatattgttgaacgctgacacctgatacttgatttcctttttcatctgcaaccttaaccaaatcagggcgaacatctaaagcttcttctggataatcatctacatagtagaaaccgttttggattgcttccttagaagatttatcgcccttatattgatccaaatcccatgttaattcgtagtagttggttgaaccagctaaaacttcttttccgtcaatattaagaccttctttattcttatttactttcgttggcttaatatagttgttatttggattatcaggatcatttggtttaccaggagtagttacacgaacaacatttgacttaatgccataagcatcattgactgtcaaagtaaagttattagtataagttgctccatcattcaatactcgaccaacaaccgttggatgaagagtctcaacaggtttagttaagtcagcattgtatgttgccaatgtctcatcagtcgccttaaaggttacagtatgactggcttcatcataagttgtatcaaagcctgtacttgcagccttagttgcatccaagtcaaacttatagccagttggaagcggatctaccaatacaaatgaagtagtttttggacgtccagctgtcaaagcttcagttttcaattcaaacttgacaattgactgtttagcaaccaatgtccggtcaatatctacaccatcctcattcttgatttctttattaatctgaggctgagctaataaattgctatagtggaaatgaactgtcggtacaactggtggagttggtaactgcttaggagttggtgctagcggtggagttggcaatggatcatatgttggctcttccggttttgatggctccggttgatctggagtcttaactggtggagttggctcattttcatatgttggtgctactggtgctggctccaatggcttctctgtctcatatgttggctcttccggttttgacggctccggttgatctggagtcttaactggcggagttggctcattttcatatgtcggtactactggtgctggctccaatggtttctctgtctcatatgttggctcttccggttttgacggctccggttggtctggagtcttaactggtggagttggctcattttcatatgtcggtgctaccggtgctggctccaatggtttttccacttcataagtaggagcttgtggttcagttggtgctaccggcggagttggtttttccttagtaattttcggaacgttaacagcacgaatcttaccattgagcgagtaccagatgtttggtcttttaccttcggtattgtctcttccaggtacttgaggcatttctgctacagacatcacattggtagcagatgttgcaccaactgtaacgtgattcgtaggaccggacatactaatggcccctgcaccgtaccaagagtttggtgcatctgatgaatcccaacctgaaccaggttcatttgctctaggatacatagtccaacgagatccaccctgatcttttctaaaattgagggttttggtagcataaatcatgccatttttttcaccgatggatgaacctgagattttgataaaagtaccactataatccttggccatctcaatagagttatgctcacggttaagtgaggctacagaaagaagggcgttgtcaaaattaattggctgatcattttcatcatagaaggtgaattcatttttaatgaaaagcgaagtatctttttctacttgaccagtataggctgaagcaaagactcctagagtagggtcggtaaagattcccaaccaaacttttccactaggatttttaaattgcgaagaaggatctacagtatatttgtaaacaatcttcgaaatctttttaccattgtagtaggaattttgaaggttggtataagtcgctgttacgctttgaccacgttctagaagaacagatccccatttaacttcagttttattccctacattggtggtccagtcagatttgtcgttgatgttaccatacaattccatagaagtgctagttgcagctgctccttctaaataagtaacgtttagattatctaactgaagattctttttaccatattgcgctgcatcttgtttaaaagcattatctacagccgttgctttgagaaagcttccttcagttaccaaagatagttgagcatttggttctgagtcatacactaagctttgagcagatggtttagacaagtatccatcttgatctttattcttttcaagttctacaagtgccgccttaattttggcctgctcatcttcataagccttcaacttagtcggatactcagccaactctttcttatatttggcaagatctgcttcatatttagctaatttggcttcatagtcagttttagctgcggcattacgttgcttgatttcttcgttttcagcttggagcgctgcattttttgccttgttgtcttcaacggccttctcataagctgctttagcatccgcatttgccttttgaacacgagccaattccgtctgatattcggctagcttagcttgatagtctgcttcactgtcttcgttggctttctttactgctgccaaatctgcttcatattgcttcaatgctgcttcataagtcgctttagctgcggcattccgttgcttaatggcttcattttcagcttgaatggctgtatttttagctgtgttttcttcaacagctttttcataggctgctttagcatcagcatttgccttttgaacacgtgccaactccgcttggtaagctgctaattttgcttggtagtcagcatcattatcttccttagcttttttaatagctgcaagatctgcttcatactgcttcatcgctgcgtcatagtttgctttggcagtttcattgcgttgcttaatcgcttcattttcagcttggagtgcagcatttttagcagtattttcctttactgctttttcataagcttctttagcttcagcattcgctttttgaacacgagccaattctgcttggtaagctgaaagcttattttgataatcaagctgactgtcttcattagctttttgaacagcggctaaatctttttgatattgagctagcttcgcttcatattctgcctttgctgtcgcattagcattgttgattttttcaacttcagcttgatgagctttcaaatcttcttgatacttatcttcagcagctttgttttcagcattgattttatctgtttcagcctgatgagcctctacttcttttttataagcttcagttgttttctttatttcttctgcttgtttagcgtaatcactcttgatttcagcttgcttttgcgcattttctgcggcagttgtggctgtacctttatcgcttgtttgatcttgtacaacattgacacctgctgctttggcatccgtcactgctttatttaaatcatcagcagatacttctactggcagagcaccatctttagaaccagcctgtgcttgactttggcttgctgcttcagtcgcttctccctgtgcttcaggcagattagtagctggatttccagttgtagtgactgctacatttgctgtactatttgtttctgtaacttcatcagccaaaacttgctgatctgcaatagcaatcaaagccgcccctaagacagctccacataaagtcttagcgactttacttttacgaaaaccaaaaacttcttttcttttgttcat

SEQ ID NO: 6 - protein, 948 aa, Actinomyces viscosus
MLRRFFVRSHRRELTSSRQPSRQSRRRLRSGAAISTFALIAGALGTATLPAPPAEAVHGNPAGGSGKYTQVIDWIDWTEMTNTLPGKGTRILPDGSTGVVWSTPSRISGDKWRTSRCTLSNVRTTAVGKNETGLPTDRGLSVGYYPGNWRGDGLARLYNDGTNYTAGVAKKPYATSSNLPLGIANLRDSSTQQFQIECSAYLVTSASQPAKSQLEHFPNKVEVPMEGIVFADAEASSWHIPNGQKEYISVSPIPYVASQNVTYRLLESARPPGCITNSVVGKVSVSTPVGNRPGVKLRPDDAECSAKVPNGYGPSSVMLLSNMRTGYVEIHGGGRGAVAFGVVSYMDYGDAPESYGVAGSAFQPAWSGGELSDSGERNIARGKAPYEGDAGDWYNLSAATDQGNVATTNSAVVRLGALTDHDDTITHSADASRDNTTDIDDEDALPAGWDRVIWTDIGQKWSQEITCSGSNTKIAGWVDWNRDGTFSAGERSAVTSCSSAGKATVTWTVPQDAKRSTINGNGAATFMRLRITGAPPNGREAEDPQPTGIAVNGEVEDHQVQVQLPNLSLAKQVDNTAAGSLGLSAKDWTLTATPKRGKTASGAGGFDSAYLPQGQTVLSESSSSPKSAGYKASVSCVPHPNSDIRTPSSTVNSASKTLDLATGEWMQCTMVNTAQPGQVVWSKVDDAGNPLAGTVFTLASPALNGGQKEVTDCVVTGGKATCPNGSVDQDPRAGFFRVSGLTWGNYSITETQAPAGYHLSSTTLTKTLDGSAPAAGTDDDTPTLDLGQVTNTRIKGSATWTKTDERGNPIKGAQWSLVPLDSNGRPQPDQARTITDCVGTCAQGSLDTDGNPGAFKLAELGYGSYGLMETKPPTGYILDATPRTITISSQGQVVALGKISNRKSAVPAIPFTGGSAADTFLIAGGAVLGITAPVMMIQAYRRRRALDS